(19) United States
(12) Patent Application Publication
(10) Pub. No.: US 2014/0360862 A1
(43) Pub. Date: Dec. 11, 2014

(54) GAS TREATMENT USING SURFACE PLASMA AND DEVICES THEREFROM

(71) Applicant: OLD DOMINION UNIVERSITY RESEARCH FOUNDATION, Norfolk, VA (US)

(72) Inventors: Muhammad Arif Malik, Norfolk, VA (US); Karl H. Schoenbach, Norfolk, VA (US)

(21) Appl. No.: 14/362,181

(22) PCT Filed: Dec. 3, 2012
(86) PCT No.: PCT/US12/67606

§ 371 Date: Jun. 2, 2014

Related U.S. Application Data
(60) Provisional application No. 61/566,372, filed on Dec. 2, 2011.

(51) Int. Cl. B01J 12/00; G21F 9/02; H05B 6/46; B01J 19/08; C07C 37/00

(52) U.S. Cl. CPC B01J 12/002; B01J 19/088; C07C 37/00; H05B 6/46; G21F 9/02; B01J 2219/0809; B01J 2219/0849; B01J 2219/0875
USPC 204/165; 204/164; 34/246; 219/778; 422/186.04; 588/18

(57) ABSTRACT

Gas treatment systems and methods are provided. A system includes at least one device defining a space and having a gas inlet and a gas outlet. The device also includes an electrode assembly, where the electrode assembly includes a dielectric plate, at least one first electrode, at least one second electrode, and a conductive layer. The electrodes are elongate electrodes disposed on a first major surface of the dielectric plate and arranged substantially in parallel. Further, the conductive layer extends over a second major surface of the dielectric plate, is electrically coupled to the one of the electrodes, and is electrically isolated from the other electrode. The system includes a circuit configured for generating a pulsed electric field between the electrodes.

### CROSS REFERENCE TO RELATED APPLICATIONS

This application claims the priority U.S. Provisional Patent Application No. 61/566,372 filed Dec. 2, 2011, the contents of which are hereby incorporated in their entirety.

### FIELD OF THE INVENTION

The invention relates to devices and methods for chemical processing. More specifically, the invention relates to energy efficient and scalable device based on non-equilibrium (non-thermal) plasma for the treatment of a gas, including steam, at atmospheric pressure without the need for diluting the steam with some inert carrier gas by the use of a surface plasma reactor.

### BACKGROUND

Plasma formation in steam is generally difficult due to the rate of electron attachment by water molecules being higher than other molecular gases, and dissociative recombination of H₃0⁺ with electrons. Consequently, plasma in steam is typically not stable, the current flowing through the plasma is typically low, and the rates of chemical reactions initiated by the high energy electrons are also typically low. A summary of different approaches through which plasma in steam has been stabilized by different research groups in the past is the following: (1) dilute the water vapor with inert gas, as plasma is easier to form in inert gases; (2) employ below atmospheric pressure in the discharge chamber; (3) allow plasma channels (streamers) to transition into arc, as the arc draws huge current; and (4) form plasma in close proximity to a dielectric surface, as additional electrons emitted by the surface through photo- or thermionic emission.

The first two approaches are not preferred in industrial applications due to low throughput and added complications due to the requirement of an inert gas supply or vacuum system. The third approach, i.e., arc discharge, is thermal plasma where the temperature of the working gas is extremely high and significant heat losses occur. Due to very high temperature of gas, the arc discharge cannot be used for partial oxidation reactions, such as conversion of hydrocarbons in fuels into oxygen containing organic compounds like aldehydes, ketones, alcohols, carboxylic acids or nitrogen containing organic compounds. The fourth approach, i.e., plasma in close proximity to dielectric surface, allows atmospheric pressure non-equilibrium plasma formation in pure water vapor but energy density in the plasma still needs to be increased for treating large volumes relevant to practical applications.

### SUMMARY

In a first embodiment of the invention, a method for the treatment of a gas is provided. The method includes providing the at least one device including first and second dielectric plates facing each other and defining a discharge region, at least one first electrode, at least one second electrode, and a conductive layer, the at least one first electrode and the at least one second electrode each including elongate electrodes disposed on an inner surface of the first dielectric plate and arranged substantially in parallel, the conductive layer disposed beneath the inner surface and extending over at least the portion of the first dielectric plate between the at least one first electrode and the at least one second electrode, and the conductive layer electrically coupled to the at least one second electrode and electrically isolated from the at least one first electrode. The method also includes directing a first gas into at least one device, generating a plurality of voltage pulses between the at least one first electrode and the at least one second electrode to generate a substantially non-thermal plasma in a first gas in the discharge region to yield a second gas, and directing the second gas from the at least one device. In the method, the generating includes selecting a voltage, a repetition rate, and a pulse width for the plurality of voltage pulses based on a thickness and a permittivity of the first dielectric plate and a gap between the at least one first electrode and the at least one second electrode.

In the method, the providing includes selecting the thickness of the first dielectric plate to be between 1 μm and 1 cm.

In the method, the generating can also include selecting the voltage for the plurality of voltage pulses to be between about 100V and about 300 kV, such as between about 10 kV and about 50 kV, or about 30 kV.

In the method, the generating can further include selecting the pulse repetition rate to be between about 1 Hz and about 10000 Hz, such as between about 200 Hz and about 500 Hz, or about 250 Hz.

In the method, the generating can also include selecting the pulse width to be between about 1 ns and about 1000 ns, such as about 150 ns.

The first gas can be contaminated air or a mixture of air with other gases, where the contaminant in the contaminated air is one of a toxic volatile organic compound, a biological agent, or an odor-causing compound.

In one configuration, the method can include selecting the first gas to be a mixture of steam and benzene, selecting the plurality of voltage pulses to cause the non-thermal plasma to generate radicals from the steam that react with at least a portion of the benzene to produce phenol in the second gas, condensing the second gas to generate a liquid, distilling the liquid to separate liquid phenol and a third gas including steam and benzene. Further, the method can include directing the third gas into the at least one device.

In another configuration, the method can include selecting the first gas to be a mixture of tritium-contaminated heavy water molecules and hydrogen containing deuterium, and selecting the plurality of voltage pulses to cause the non-thermal plasma to result in a hydrogen isotopic exchange between the tritium-contaminated heavy water molecules and the hydrogen containing deuterium.

A second embodiment of the invention provides a system. The system includes at least one device and a circuit. The at least one device includes one or more dielectric portions defining at least one elongate and substantially continuous inner surface with an inlet and an outlet, at least one first electrode, at least one second electrode, and at least one a conductive layer, the at least one first electrode and the at least one second electrode disposed on the inner surface and the at least one conductive layer beneath the inner surface and substantially surrounding a discharge region defined by the inner surface, the at least one conductive layer electrically coupled to the at least one second electrode and electrically isolated from the at least one first electrode. In the system, the circuit is in communication with the at least one device and configured for generating a plurality voltage pulses between the at least one first electrode and the at least one second electrode.

In the system, each of the at least one first electrode and the at least one second electrode can be elongate electrodes disposed on the inner surface of a tubular dielectric parallel to each other. Further, the elongate electrodes can extend substantially parallel to an axial direction of the inner surface. The elongate electrodes can alternatively be disposed at the inlet and the outlet of the tubular dielectric.

In some configurations, the inner surface can be a substantially cylindrical surface.

In other configurations, the at least one device can be a plurality of devices, where a first of the plurality of devices is disposed with the discharge region of a second of the plurality of devices, and where the at least one conductive layer of the second of the plurality of devices is not exposed to the discharge region of the first of the plurality of devices.

In still other configurations, the at least one device includes a plurality of devices, and the discharge region of first of the plurality of devices is connected in parallel with the discharge region of a second of the plurality of devices. Alternatively, the discharge region of first of the plurality of devices is connected in series with the discharge region of a second of the plurality of devices.

In a third embodiment of the invention, a system for the treatment of a surface is provided. The system can include a first dielectric portion with a first inner surface and a first outer surface. The system can also include a second dielectric portion with a second inner surface and a second outer surface, the second dielectric portion disposed adjacent to the first dielectric portion such that the first inner surface faces the second inner surface and defines a discharge region. Further, the system can include a first electrode disposed at an inlet end of the discharge region on the first inner surface, and at least one second electrode disposed at an outlet end of the discharge region, the at least one second electrode disposed on at least one of the first inner surface and the second inner surface. Additionally, the system can include at least one conductive layer extending over the first outer surface and the second outer surface, where the at least one conductive layer electrically coupled to the at least one second electrode and electrically isolated from the at least one first electrode. Finally, the system can include a circuit in communication with the at least one device and configured for applying a series of voltage pulses between the at least one first electrode and the at least one second electrode.

In one configuration, the system can include a slit cover coupled to the outlet end having at least one slit. In some cases, the at least one slit has a length between 1 cm and 10 cm and a width between 0.01 cm and 1 cm. In some cases, the at least one slit includes a plurality of slits.

In another configuration, the first dielectric portion and the second dielectric portion can be substantially rectangular dielectric plates, the dielectric plates arranged substantially in parallel and substantially overlapping each other. Further a spacing of the dielectric plates can be between 0.01 cm and 1 cm.

The system can further including a source of air or other gases or mixtures thereof coupled to the inlet end. Alternatively, the system can include a source of steam coupled to the inlet end.

In the system, the voltage pulses delivered by the circuit can be between 100V and 500 kV. Further, the voltage pulses delivered by the circuit can be delivered with a repetition rate between 1 Hz and 1000 Hz.

In a fourth embodiment of the invention, a method of treatment of a surface is provided. The method includes providing a device including at least one dielectric portion defining discharge region with an inlet and an outlet, at least one first electrode, at least one second electrode, and at least one a conductive layer, the at least one first electrode and the at least one second electrode disposed on a major surfaces of the dielectric portions facing the discharge region, the at least one conductive layer substantially surrounding the discharge region and electrically coupled to the at least one second electrode and electrically isolated from the at least one first electrode. The method also includes directing a gas into the inlet, generating a plurality of voltage pulses between the at least one first electrode and the at least one second electrode to generate an activated gas, and applying the gas at the outlet against a surface to be treated. In the method, the plurality of voltage pulses can be selected to generate a corona discharge primarily including surface streamers.

In some configurations, the gas can be air, steam, a mixture of air with other gases, or a mixture of steam with other gases.

In some configurations, the device further including a slit cover coupled to the outlet end of the device and including at least one slit. Thus, the method further includes exposing the surface to be treated to gas at the outlet via the at least one slit.

In the method, the generating further can include selecting the plurality of voltage pulses to be between 100V and 500 kV. The generating further can include selecting the plurality of voltage pulses to be applied with a repetition rate between 1 Hz and 1000 Hz.

### BRIEF DESCRIPTION OF THE DRAWINGS

FIG. 1A shows an exemplary schematic of a reactor configured in accordance with an embodiment of the invention.

FIG. 1B shows a cross-section view of reactor through cutline 1-1 in FIG. 1A.

FIGS. 2A and 2B show electric equipotential line plots for electrodes in space and electrodes arranged as in FIGS. 1A-1B.

FIGS. 3A and 3B show photographs of the sliding discharge for the configurations of FIGS. 2B and 2A, respectively.

FIGS. 4A and 4B shows an exemplary schematic of multiple electrode assemblies configured in accordance with an embodiment of the invention.

FIG. 5A is a series of photographs showing plasma discharges and discharge data for systems in accordance with the various embodiments with various inter-electrode gaps and an effective electrode length of 50 mm.

FIG. 5B is a photograph showing plasma discharge and discharge data for a system in accordance with the various embodiments with an inter-electrode gap of 5 mm and an effective electrode length of 360 mm.

FIG. 6A is an illustration of a single discharge chamber for tritium extraction from tritium contaminated heavy water from nuclear power plants or other tritium contaminated water sources by atmospheric pressure non-equilibrium plasma in steam and hydrogen mixture.

FIG. 6B is an illustration of a single discharge chamber for phenol synthesis from steam and benzene.

FIG. 7A is a schematic of the experimental setup showing an electrode assembly in the reaction vessel for the experimental setup with a power supply and a condenser connected.

FIG. 7B shows a detailed view of the electrode assembly for the experimental setup.

FIG. 7C shows a cross-section of the electrode assembly along 1-1 line in FIG. 7B.

FIG. 8A shows an electrode configuration for conventional pulsed corona configuration that discharges in steam.

FIG. 8B shows an electrode configuration for discharging in steam using plasma in contact with dielectric surface, but with no conductive layer on a second major surface.

FIG. 8C shows an electrode configuration in accordance with an embodiment of the invention.

FIG. 8D shows stacked electrode configuration in accordance with the an embodiment of the invention.

FIG. 9 is a time integrated image of atmospheric pressure non-equilibrium plasma in steam in the case of the electrode assembly of FIG. 8C.

FIG. 10A is an x-y plot of the production of hydrogen and oxygen with a single reactor (such as shown in FIG. 8C).

FIG. 10B is an x-y plot of the production of hydrogen and oxygen with two reactors operating in parallel (such as shown in FIG. 8D).

FIGS. 11A, 11B, and 11C show schematically the experimental setup for one, two, and three surface plasmas in a tubular or cylindrical configuration without shield, respectively.

FIGS. 12A-12C schematically illustrate the experimental setup showing the effects on the plasma formation in the neighboring chambers in tubular or cylindrical configuration when they are operated in parallel and are separated by shield portions.

FIG. 13 is a x-y plot of hydrogen generation as a function of power for single and dual discharge chamber systems in accordance with the various embodiments.

FIG. 14 is a x-y plot of hydrogen generation as a function of time, dielectric material, and thickness for discharge chamber systems in accordance with the various embodiments.

FIG. 15 is a x-y plot of energy yield, with respect to hydrogen generation, as a function of time, dielectric material, and thickness for discharge chamber systems in accordance with the various embodiments.

FIG. 16 is a x-y plot of hydrogen peroxide concentration as a function of time, dielectric material, and thickness for discharge chamber systems in accordance with the various embodiments.

FIG. 17 is a x-y plot of energy yield, with respect to hydrogen peroxide generation, as a function of time, dielectric material, and thickness for discharge chamber systems in accordance with the various embodiments.

FIG. 18 is an x-y plot of absorbance as a function of wavelength for different treatment times using a plasma generated in steam+benzene mixture in accordance with the various embodiments.

FIG. 19 is an x-y plot of NO removal (ppm) from air as a function of input energy for various configurations of a system in accordance with the various embodiments and different electrode polarities.

FIG. 20 shows an exemplary schematic of a plasma discharge chamber in accordance with an alternate embodiment of the invention.

FIGS. 21A and 21B show photographic image of plasma in a large slit configuration and a small slit configuration, respectively, of the system of FIG. 20.

FIGS. 22A and 22B show cathode directed streamers and anode directed streamers, respectively, of the system of FIG. 20.

FIG. 23 is a x-y plot of voltage and current waveforms and cumulative energy per pulse in the plasma reactor with air flowing at the rate of 20 liters per minute.

FIG. 24 is a plot of ozone concentration with discharge slit 2.6 cm×0.038 cm.

FIGS. 25A-25C show treatment results for various large slit configurations.

FIGS. 26A-26C show treatment results for various small slit configurations.

FIG. 27 shows bacterial Log₁₀ recovery on entire plate for various large slit configurations.

FIG. 28 shows Bacterial Log₁₀ Recovery on treatment area for various large slit configurations.

FIG. 29 shows bacterial inactivation results for entire plate various large slit configurations.

FIG. 30 shows Bacterial Log₁₀ Recovery on treatment area for various large slit configurations.

FIGS. 31A-31C show alternative system configurations in accordance with the various embodiments of the invention.

FIG. 32 illustrates a system including a gas treatment device, configured in accordance with an embodiment of the invention, and supporting electrical circuitry.

FIG. 33 is a detailed block diagram of a computing device which can be implemented as a control system.

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

Further, various non-limiting examples and exemplary results will be presented throughout that serve to illustrate selected embodiments of the invention. It will be appreciated that variations in proportions and alternatives in elements of the components shown will be apparent to those skilled in the art and are within the scope of embodiments of the invention.

The terms “substantially” as used herein with respect to a value, refer to being within 20% of the stated value. As used herein with respect to a condition or property, the term “substantially” refers to matching the stated condition or property to a large extent.

Additionally, although the various embodiments will be described with respect to the treatment of specific gases using specific species, the various embodiments are not limited in this regard. Rather, the various embodiments can be adapted for the treatment of any other types of gases or combinations of gases (e.g., air). As such, the specific example of gases and combinations thereof are provided solely for ease of illustration and not by way of limitation.

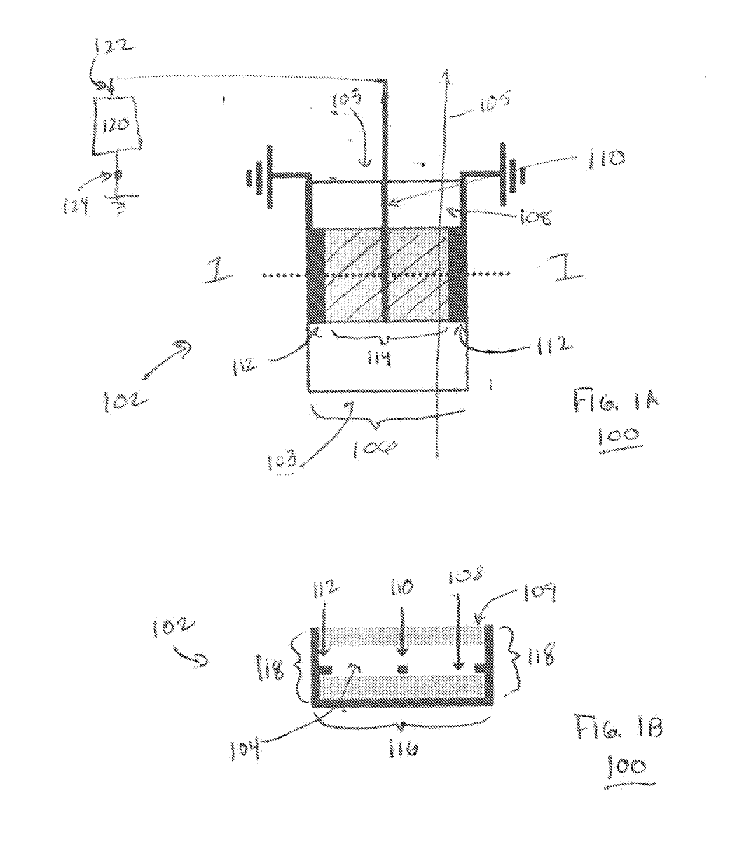

FIG. 1A shows an exemplary schematic of a system 100 configured in accordance with an embodiment of the invention. FIG. 1B shows a cross-section view of gas treatment system 100 through cutline 1-1 in FIG. 1A. As shown in FIGS. 1A and 1B, the system 100 includes a reactor 102 defining a discharge chamber or space 104. In the various embodiments, the system 100 and other systems described herein is designed to operate primarily with a pressure that is substantially atmospheric pressure. As used herein, the term “approximately atmospheric pressure” refers to a pressure at or near 1000 mbar (i.e., −1 atmosphere), including pressures in the range of about 100 mbar to about 10,000 mbar, such between about 500 mbar and about 2000 mbar.

The reactor 102 can include gas inlet/outlet portions 103 to allow gases to travel through space 104, as indicated by direction 105. In the various embodiments, gas inlet/outlet portions 103 can be configured in a variety of ways with respect to reactor 102. For example, gas inlet/outlet portions 103 can be configured based on the source or destination of the gases.

In one exemplary configuration of system 100, the reactor 102 can be constructed using glass, acrylic, or other dielectric materials. For example, the reactor 102 can be fabricated from ceramic, such as cordierite, silicon carbide, or alumina, to name a few. However, the various embodiments are not limited in this regard and other types of dielectric materials can be used. Further, although reactor 102 is illustrated in FIGS. 1A and 1B in the form of a rectangular assembly, the various embodiments are not limited in this regard. Rather, the enclosure can have any geometry, including non-rectangular geometries. Further, in addition to non-rectangular geometries, the reactor 102 can also be defined by an opening, hole, passage, or the like in a larger mass of material.

System 100 further includes an electrode assembly 106 disposed in the space 104. The electrode assembly 106 includes a first dielectric sheet or plate 108, a second dielectric sheet or plate 109, an anode electrode 110, at least one cathode electrode 112, and a conductive layer 116. As shown in FIGS. 1A and 1B, the anode electrode 110 and the cathode electrode(s) 112 are disposed on a first major surface of the first dielectric plate 108. Specifically an inner surface of the first dielectric plate 108 with respect to space 104. As used herein with respect to an object being a sheet, plate, or the like, the term “major surface” refers to either of the opposing largest surfaces of the object. The anode electrode 110 and the cathode electrode(s) 112 can be configured so that they are elongate electrodes that are substantially in parallel and that substantially coextend. That is, the anode electrode 110 substantially overlaps the entire length of the cathode electrode(s) 112 on first dielectric plate 108. Theses coextending portions of anode electrode 110 and cathode electrode(s) 112 define a glide region 114 there between where the concentration of the non-equilibrium plasma will be the greatest. In the case where two cathode electrodes 112 are provided, as shown in FIGS. 1A and 1B, the anode electrode 110 can be disposed between the two cathode electrodes 112. Further, the anode electrode 110 preferably is, but is not required to be, equidistant from the cathode electrodes 112.

As shown in FIGS. 1A and 1B, the anode electrode 110 is shown as a wire inserted into and extending across the length of discharge chamber 102. However, the various embodiments are not limited in this regard. For example, anode electrode 110 may also be a thin metal strip, threaded rod, sharp edge, or any other localizing configuration of electrode capable of producing streamers, without limitation. Cathode electrode(s) 112 can be configured in substantially the same way. However, the cathode electrode(s) 112 can also be in the form of a wire mesh, a plate, a wire, or other conductive electrode configuration known in the art.

In one exemplary configuration of electrode assembly 106, it can be constructed using substantially flat or planar sheets or films, consisting of glass, acrylic, or other dielectric materials, as dielectric plates 108 and 109. As used herein with respect to a measure, property, or the like, the term “substantially” means being within 20% of the stated value. A stainless steel wire can provide anode electrode 110 and aluminum strips can provide cathode electrode(s) 112. However, the various embodiments are not limited to the exemplary materials described above. For example, dielectric surface 104 can be fabricated from ceramic sheets, such as machineable glass ceramics, cordierite, silicon carbide, or alumina, to name a few. An exemplary machineable glass ceramic is MACOR machineable glass ceramic available from Corning Incorporated of Corning, N.Y. The dielectric can also consist of coating e.g. deposited onto the conductive layer through a sputtering process. Further, the electrodes 110 and 112 can be fabricated from any electrically conducting or semi-conducting materials. However, metals, such as stainless steel, copper, silver, tungsten, or alloys thereof would provide superior performance. Further, the materials can be selected so as to provide little or no reactivity with the gases being treated.

Further, the various embodiments are not limited to the wire-to-strip configuration of FIGS. 1A and 1B. Thus, the anode electrode 110 and cathode electrode(s) 112 can be arranged in a edge-to-edge, a wire-to-wire configuration, a point-to-wire configuration, or a point-to-plate configuration, to name a few.

Further, the roles of the electrodes in the various embodiments can be reversed. That is, anode electrode 110 and cathode electrode(s) 112 can be switched to provide a cathode electrode at electrode 110 and anode electrodes at electrodes 112.

In addition to the dielectric plates 108 and 109 and the electrodes 110 and 112 and as noted above, the electrode assembly 106 also includes a conductive region 116 disposed on a second major surface or an outer surface (with respect to space 104) of the dielectric plate 108 and electrically coupled to the cathode electrode(s) 112. As shown in FIG. 1B, the electrical coupling can be by way of one or more connecting portions 118 disposed along the edges of the dielectric plate 108 to contact the cathode electrode(s) 112. However, the various embodiments are not limited in this regard. For example, in some embodiments, the connecting portions 118 can extend through the dielectric plate 106 to electrically connect the cathode electrode(s) 112 to the conductive region 116. Further, the various embodiments are not limited to providing a discrete conductive region 116, connecting portions 118, and cathode electrode(s) 112. Rather, they can be integrally formed.

In some embodiments, the conductive region 116 can be configured to extend over substantially the entire second major surface of the dielectric plate 106. However, the various embodiments are not limited in this regard. For example, as shown in FIG. 1A, the conductive region 116 can be configured to extend to overlie at least the glide region 114. That is, over a portion of the second major surface opposite to the glide region 114.

In the various embodiments, the conductive region 116 can be fabricated from any electrically conducting or semi-conducting materials. However, metals, such as stainless steel, copper, silver, tungsten, or alloys thereof would provide superior performance. Further, the materials for the conductive region 116, the connector portions 118, and the cathode electrode(s) 112 can be the same or different.

In addition to reactor 102 and electrode assembly 106, the system 100 can further include a power supply system or circuit 120 for applying a pulsed electric field between anode electrode 110 and cathode electrode(s) 112. In particular, a first output terminal 122 of the circuit 120 can be electrically coupled to the anode electrode 110 and the second output terminal 124 of the circuit 120 can be electrically coupled to the cathode electrode(s) 112. The circuit 120 can be configured to provide a pulse electric field by providing a series of voltage pulses. The voltage pulses can be configured to have a voltage between about 100V and about 50 kV, such as between about 10 kV and about 50 kV. In one configuration a voltage of about 30 kV can be used. The voltage pulses can be applied using a pulse repetition rate that is between about 1 Hz and 10000 Hz, such as between about 50 Hz and 500 Hz. In one configuration the pulse repetition rate can be about 250 Hz. Further, the voltage pulses can have a pulse width between about 1 ns and 1000 ns, such as between about 100 ns and 200 ns. In one configuration, the pulse width can be about 150 ns. However, the various embodiments can be utilized with other parameters for the voltage pulses, depending on the application.

In the various embodiments, the electrical coupling of the second output terminal 124 to the cathode electrode(s) 112 need not be direct. Rather, as shown in FIG. 1A, the second output terminal 124 and the cathode electrode(s) 112 can be electrically coupled to a same low voltage or reference node, such as a ground node. Further, the schematic illustrated in FIG. 1A is simplified for ease of explanation. Accordingly, one or more additional elements can be provided to connect circuit 120 to electrode assembly 106.

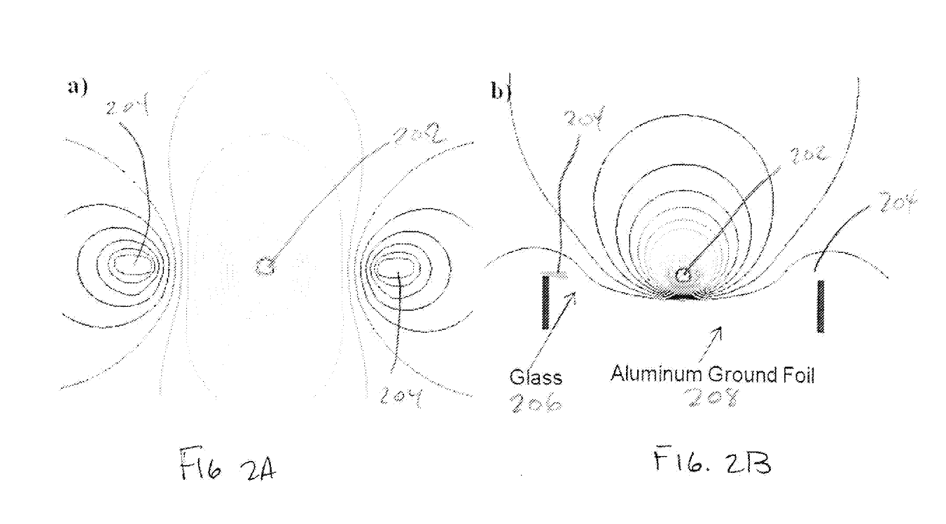

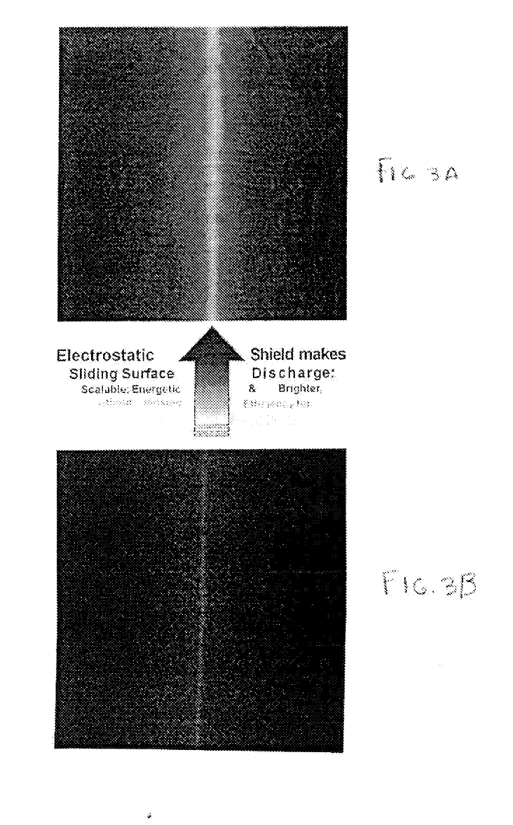

The advantages of the configuration of the electrode assembly of FIGS. 1A and 1B are shown in FIGS. 2A and 2B. FIG. 2A shows electric equipotential lines for an arrangement of a first electrode 202 disposed between two second electrode 204, similar to that of FIGS. 1A and 1B, but without a dielectric sheet or a ground plane or shield. FIG. 2B shows electric equipotential lines for an arrangement of a first electrode 202 disposed between two second electrode 204, similar to that of FIGS. 1A and 1B, with a dielectric sheet 206 and a ground plane or shield 208. As shown in FIGS. 2A and 2B, the presence of the dielectric sheet 206 and the shield 208 significantly affects the electric field (perpendicular to the equipotential lines). In particular, the electric fields between the first electrode 202 and the second electrode are significantly higher throughout the space between them in the presence of the dielectric sheet 206 and the shield 208. As such, conditions for forming surface streamers are more favorable. This effect is also visually observable. FIGS. 3A and 3B show photographs of the plasma glow for the configurations in FIGS. 2B and 2A, respectively. As shown in FIG. 3A, the presence of the dielectric sheet 206 and the shield 208 results in a brighter discharge, indicating a higher energy and efficiency in the reactor.

The structure of FIGS. 1A and 1B is scalable by stacking multiple discharge changers on top of each other or sideways. This is possible because the conductive layer or shield provided between the dielectric layers defining the chambers confine the electric fields and charges within the individual chambers. This therefore prevents electrical coupling between adjacent chambers. Coupling leads to a reduction of the electrical power in the discharges, and has therefore a negative effect on the plasma efficiency. Important for high efficiency is that the shield is tightly connected to the dielectric layer in order to maximize the capacitance defined by the plasma-dielectric layer-conductive shield. Accordingly, in some embodiments, the conductive layer or shield can be embedded into the dielectric materials, or the dielectric layer can be a coating on the conductive layer.

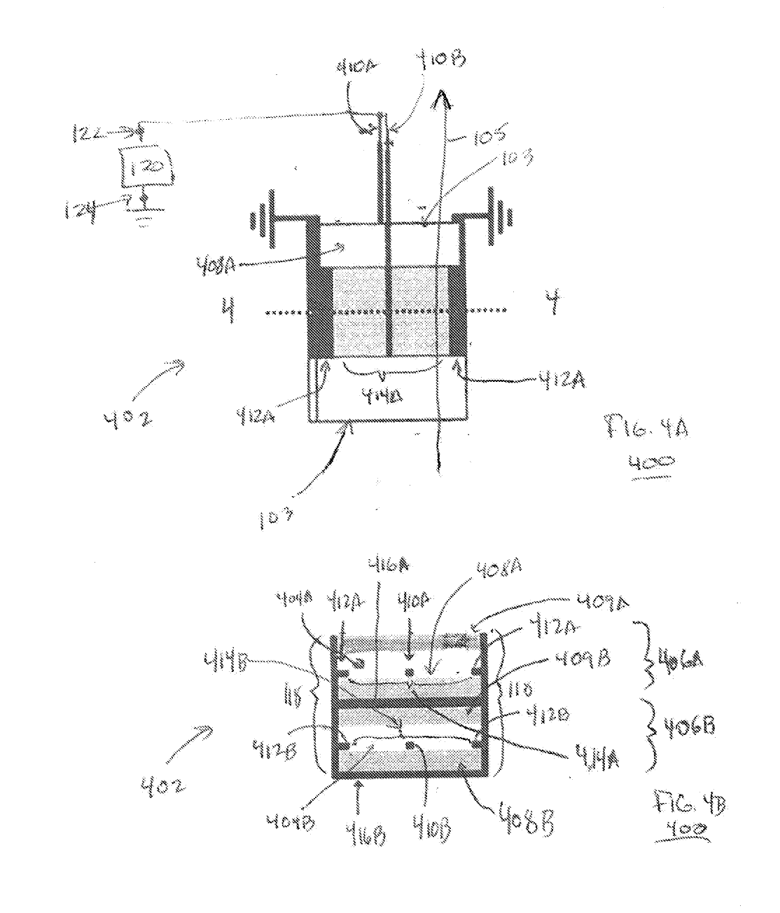

As noted above, the configuration described herein allows for the stacking or combining of multiple reactors. This configuration is illustrated with respect to FIGS. 4A and 4B.

FIG. 4A shows an exemplary schematic of a system 400 configured in accordance with an embodiment of the invention. FIG. 4B shows a cross-section view of gas treatment system 400 through cutline 4-4 in FIG. 4A. The configuration of system 400 is substantially similar to that of system 100. Accordingly, the description of system 100 above is applicable for describing system 400 except as noted below.

The main difference between system 100 and system 400 is that reactor 402 include a first electrode assembly 406A and a second electrode assembly 406B. Like the electrode assembly 106 in system 100, electrode assembly 406A also includes a first dielectric plate 408A and a second dielectric plate 409A defining a space 404A. The dielectric plate 408A is disposed on first surface of a conductive region or plate 416A, electrodes 410A and 412A disposed on the dielectric plate 408A and coupled to conductive region 416A via connecting portions 118. Similarly, electrode assembly 406B also includes a first dielectric plate 408B and a second dielectric plate 409B defining a space 404B. The dielectric plate 408B is disposed on first surface of a conductive region or plate 416B, electrodes 410B and 412B disposed on the dielectric plate 408B and coupled to conductive region 416B via connecting portions 118. As shown in FIG. 4B, these two assemblies are stacked on each other. As a result, a single connection portion 118 is effectively formed.

Thus, the arrangement of the conductive regions 416A, the connecting portions 118, and the electrodes 410A and 412A define a first glide region 414A, similar to the arrangement of components in system 100 for defining electrode assembly 106. Similarly, the arrangement of the conductive regions 416B, the connecting portions 118, and the electrodes 410B and 412B define a first glide region 414B.

As a result of the foregoing structure, a plasma with a larger treatment volume can be provided, as each glide region in each assembly will generate a separate plasma. More importantly, the plasma generated at each of glide regions 414A and 414B will operate substantially independently of each other. In particular, since the conductive region 416A (and thus electrodes 412A and 412B) are coupled to ground, the conductive region 416A effectively operates as a shield portion that prevent accumulation of charges on portions of dielectric regions 408A and 408B opposite the glide regions 414A and 414B, respectively, that could inhibit formation of plasma in either of the glide regions. Thus, the volume of plasma to effect treatment can be increased.

It is worth noting that although electrodes 410A and 410B are shown as being coupled to the same power supply in FIG. 4A, the various embodiments are not limited in this regard. Rather, electrodes 410A and 410B can be coupled to different power supplies.

The reactors described above, and specifically the configuration of electrodes, 112 and conductive region with respect to dielectric sheet, generates sliding surface discharges in the gas phase. The gas phase may be air, steam (water vapors) or mixtures thereof or any other gas. The conductive layer on the outer surface of the dielectric sheet is an extension of the electrodes and leads to re-distribution of electric field (as shown in FIG. 2B as compared to FIG. 2A) in such a way that i) the electric field at the edges of the high voltage electrodes is intensified and ii) a strong increase in the electric field component normal to the surface of the dielectric occurs.

The first effect leads to increase in the probability of plasma channels (streamers) initiation. The second effect keeps the plasma firmly attached to the surface, increasing plasma surface interaction that includes intensification of the plasma through secondary electron emissions from the surface through photo/thermionic emissions or through bombardment of charged particles on the surface. These effects allow to increase energy going into the plasma by about forty times what air is as the working gas and by about twenty times what steam (water vapors) is as the working gas, without losing efficiency for chemical reactions, such as NO conversion in the case of air or hydrogen production in the case of steam.

As a result, the design parameters for such reactors can be selected so increase their effectiveness. In particular, the design parameters can be selected to reduce the amount of voltage required for forming a particular charge on the dielectric plate, or correspondingly, to generate the same plasma. Such selection of parameters is critical since this can reduce power requirements and thus reduce the cost of manufacture and operation of a system.

There are three distinct phases of gliding discharge plasma formed using the electrode assembly described herein. First is the streamer propagation phase, i.e., starting from initiation of streamers from the high voltage electrode until the streamers reach the counter electrode. The second phase is the glow discharge phase, i.e. after bridging the inter-electrode gap, the plasma still remains non-thermal but draws a significantly higher amount of current (energy) compared to the streamer propagation phase. Since, it is still a non-thermal plasma, so it remains energy efficient for chemical reactions with the advantage of higher energy density, i.e., higher throughput. The third phase is a spark discharge, which draws a huge amount of current. But it is not desirable as it is close to a thermal plasma that is usually not energy efficient for chemical reactions, particularly not suitable for partial oxidation of hydrocarbons to obtain partially oxygenated compounds.

One parameter that can thus be adjusted is the inter-electrode gap. When the gap is small, shorter streamers are generated. The shorter streamers formed at lower applied voltages carry less energy per individual streamer and they are more efficient for utilizing the energy for chemical reactions. It is also desirable that the total energy density in the reactor not decrease as it will decrease the treatment volume (throughput). Thus, by reducing the gap length it is also possible to reduce the size of the reactor without affecting the total energy density. Further, the inter-electrode gap needs to be adjusted such that the streamer phase (streamers propagate at lower speed for lower voltage), and the glow phase coincides with the electrical pulse duration. If the electrical pulses are longer, or if the voltage is higher, a glow to spark transition would occur.

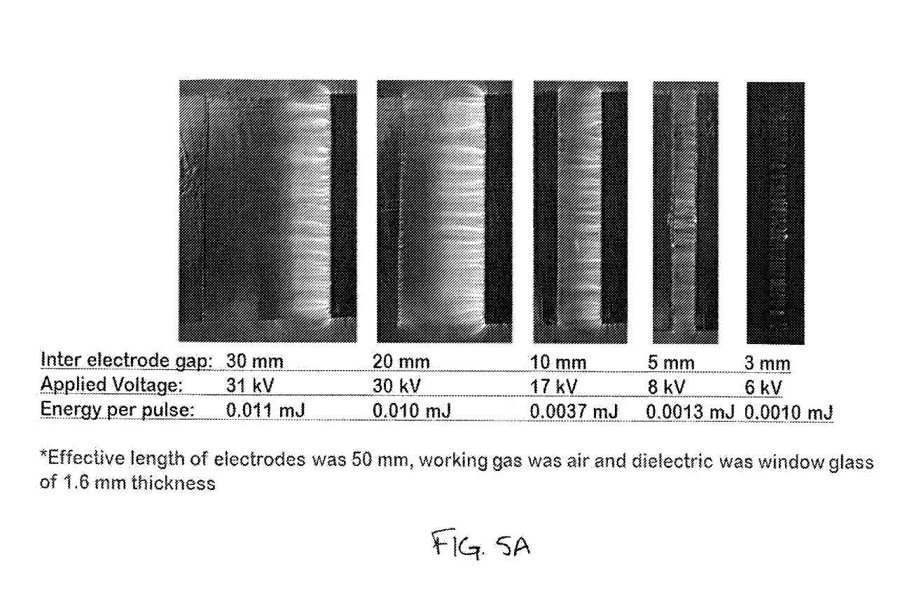

In some configurations, the reduction of the inter-electrode gap can be beneficial, but other modifications can be necessary. Referring now to FIG. 5A, FIG. 5A shows photographs of the resulting plasma discharge for an electrode arrangement in accordance with the various embodiments for various inter-electrode gaps (30 mm, 20 mm, 10 mm, 5 mm, and 3 mm), applied voltages, and resulting energy per pulse. For each of the electrode arrangements in FIG. 5A, the effective length was approximately 50 mm. Most significantly, the results in FIG. 5A show that as a result of reducing the electrode gap, the amount of voltage required to be applied to provide a plasma is reduced. Further, the amount of energy per pulse is also reduced. However, while such a configuration may reduce the amount of power, the spatial distribution of streamers is substantially reduced, thus reducing the available treatment volume. In the various embodiments, this deficiency can be made up by extending the length of the electrodes, as illustrated in FIG. 5B. FIG. 5B is a photograph of the resulting plasma discharge for an electrode arrangement with a 5 mm inter-electrode gap and an effective length of approximately 360 mm. As shown in FIG. 5B, the 5 mm gap allows the plasma discharge to be established at a voltage of 9 kV while providing an energy per of 0.0068 mJ. This provides a substantial increase in energy per pulse and treatment volume as compared to a configuration with a similar gap (0.0068 mJ versus 0.0013 mJ), but without requiring an increase in voltage applied at the electrodes.

A second parameter that can be adjusted is the capacitance. For increased capacitance dielectric plates (thin, high permittivity) streamer density can be increased and the glow discharge mode (second phase) may be reached at a lower applied voltage, especially at lower gap lengths. In the various embodiments, the thickness of the dielectric layer can be varied between 1 μm (thin layers can be obtained by placing a dielectric coating on the shielding metal plate) to 1 cm. The dielectric constant or relative permittivity can be varied from 1 to 1,000,000. For example, there are extremely high permittivity materials now available, e.g. calcium copper titanate has relative permitivities greater than 250,000.

However, the design parameters need to be selected carefully and can require adjustment of both the dielectric and the gap length. For example, as noted above, a glow discharge mode can be obtained at lower voltages as the gap length is decreased and capacitance is increased. However, if the gap length is too low, the spark mode will take over. Accordingly, a reduction in capacitance can require an optimization of gap length or vice versa to prevent entering the spark mode.

Thus, enhancements can be obtained by selection of the permittivity or thickness of the dielectric sheets or by selection of the gap between opposing electrodes. Alternatively, the configuration of the voltage pulses can be selected so that low voltage streamers are preferably formed.

As noted above, the discharge chamber geometry is not limited to that illustrated in FIGS. 1A and 1B. For example, the discharge chamber can be modified such that the basic geometry is cylindrical, as described below. In such configurations, the same concept of sliding discharges between parallel edge to edge electrodes on a dielectric layer with one electrode extended to form or coupled to a conductive. In such configurations, a gas flow can be either parallel to or perpendicular to the electrodes, where perpendicular means that it is still in the plane of the plasma layer.

Other theoretical aspects and details regarding of electrode assemblies similar to those shown in 1A and 1B and other electrode assemblies and configurations described herein are discussed in greater detail in International Publication NO. WO 2012/044875, published Apr. 5, 2012, the contents of which are herein incorporated by reference in their entirety.

The resulting reactors provided by the configurations shown in FIGS. 1A, 1B, 4A, and 4B can be used for a variety of applications. A first possible application is the extraction of radioactive tritium from coolant/moderator of nuclear power plants. In such applications, tritium is first extracted by isotopic exchange between water molecules and gaseous hydrogen. This exchange is typically enhanced by a catalytic reactor or by plasma. In general, DC discharge plasma below atmospheric pressure or micro-hollow cathode discharge plasma at atmospheric pressure has been primarily tested for this purpose.

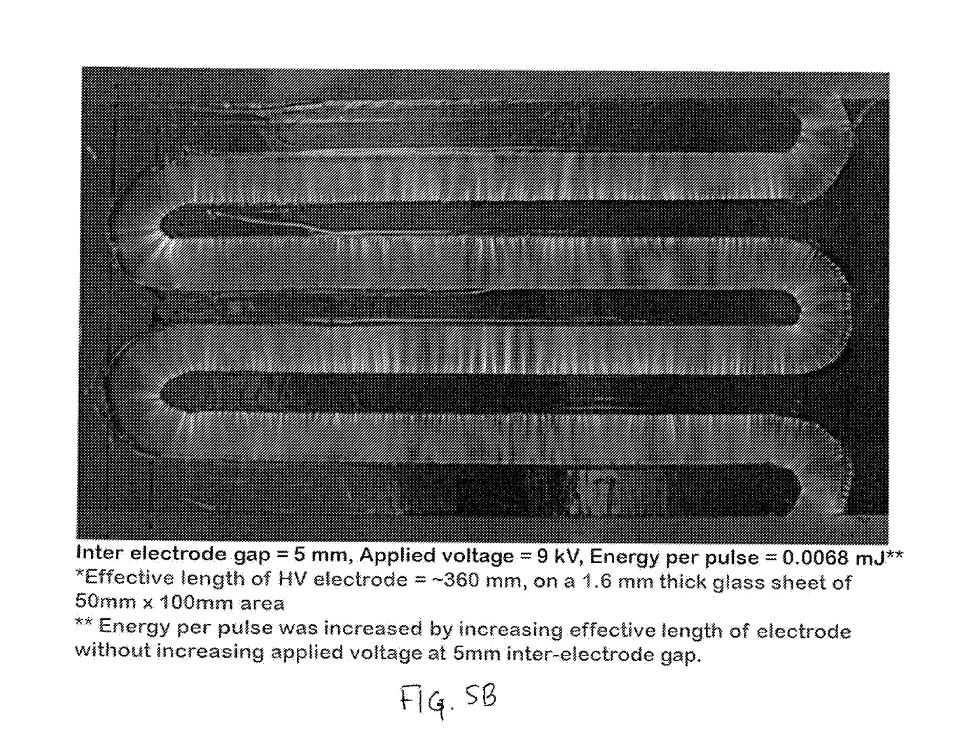

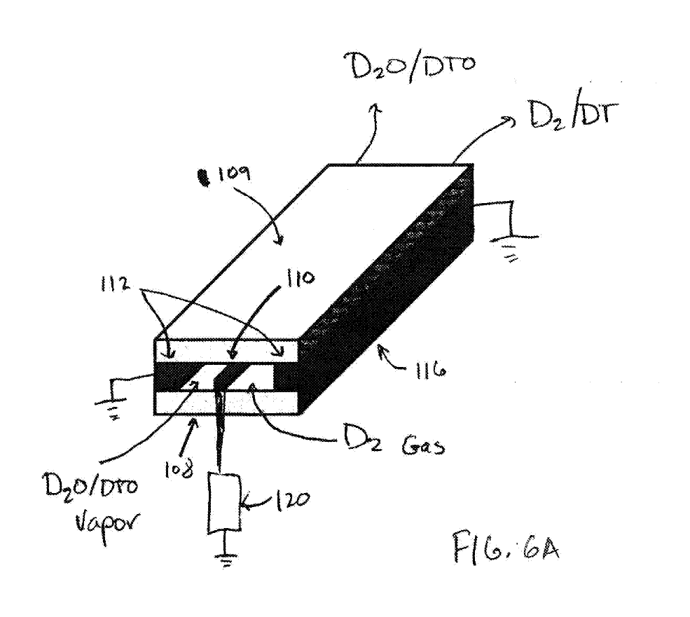

A reactor in accordance with the various embodiments allow for increasing the energy density and increasing the chemical reactions in pulsed corona discharges in steam at atmospheric pressure that can treat much larger volumes of the process gas. Further, such reactors provide dissociation of water molecules and hydrogen molecules forming atomic hydrogen, making feasible hydrogen isotope exchange between hydrogen and water molecules needed in the tritium extraction process. The extracted tritium can then be enriched by an accompanying technique of cryogenic distillation or diffusion process. The process of tritium extraction using a system as described with respect to FIGS. 1A and 1B is schematically illustrated in FIG. 6A. As shown in FIG. 6A, with the help of the example of hydrogen isotopic exchange between tritium-contaminated heavy water molecules and hydrogen containing deuterium. The isotopic exchange between water molecules and gaseous hydrogen takes place at much faster rate in plasma compared to that in conventional catalytic exchange. Use of a reactor in accordance with the various embodiments can increase energy density in the reactor can therefore process larger volumes of the working gases. Further, it allows scaling up by operating multiple reactors that are needed in industrial applications. The materials of construction of the electrode assembly can be such that they are resistant to radiations and do not leach out. It is worth noting that the processes described above are not solely limited to tritium extraction from heavy water. The processes can also be extended to tritium extraction from light water as well.

A second possible application is the steam reforming of fuels or conversion of the hydrocarbons in the fuel into oxygenated hydrocarbons, including reforming of methane, bio-gas, gasoline and diesel. Traditionally, steam reforming of fuel with non-equilibrium plasmas has been carried out by diluting the steam and fuel mixture with some inert gas or by allowing the steamers to transition to arc (i.e., a thermal plasma) or by outright use of thermal plasmas. However, in a reactor in accordance with the various embodiments, there is no need to dilute the working gas in the case of fuel reforming, which is a major advantage over other non-equilibrium plasma reactors. Further, such reactors are more energy efficient, as non-equilibrium plasma is generally more energy efficient than thermal plasma. The experimental conditions can be optimized to obtain partially oxygenated hydrocarbons, which is difficult to do with thermal plasmas. Additionally, as discussed above processing of large volumes of working gas will be possible in the proposed reactor. Also, increased rates of reaction are possible with a reactor in accordance with the various embodiments due to a larger current going through the plasma.

A third possible application is in the field of providing surface treatments. The hydroxyl radicals produced as a first step in the dissociation of water molecules (H₂0+e*→OH+H+e) are generally useful for surface treatment of polymers and other materials. The intense plasma formed in working gases other than steam will also be effective for this application.

A fourth and related application is the sterilization of surfaces and related medical applications. The plasma jet that is usually formed in tubular dielectrics and employed for sterilization of surfaces and other related medical applications is generally low intensity plasma. A reactor in accordance with the various embodiments allows the forming of more intense plasma. Also, it also allows increasing the area of the plasma zone by operating multiple plasma reactors in parallel. For example, plasma formed in multiple dielectric tubes bundled together, with the plasmas decoupled by extending the electrodes forming a conductive layer around the dielectric tube, as discussed above and below. Consequently, the increase in current going through the plasma through the multiple tubes will increase the rates of production of reactive species needed for the treatments.

A fifth possible application is the synthesis of chemical compounds by the reactions of reactive species produced by the plasma. For example, the hydroxyl radicals produced by the plasma in steam can be employed to synthesize phenol from benzene, as illustrated in FIG. 6B Synthesis of phenol from benzene by non-equilibrium plasma in liquid water has been previously reported. However, phenol reacts further as it is more reactive than the benzene. In the case of a reactor in accordance with the various embodiments, the benzene can be sent into the plasma zone by steam distillation, and the un-reacted benzene, water and the product phenol can be condensed and re-cycled. During the cyclic operation, steam and benzene distill out (via distiller 602) and can re-enter the plasma zone leaving behind the phenol product, as phenol is water soluble and does not distill out. In this way, continuous phenol production from benzene can be carried out without further oxidation of phenol. The reactor made of ceramic and metal materials can form the plasma in a wide range of temperature, e.g., from below freezing point of water to above 800° C. Operating the plasma reactor at temperatures above the boiling points of intermediates/products can eliminate the problem of deposits that otherwise usually form on the electrodes assembly. The increased rates of chemical reactions will apply to other chemical reactions as well. As a result, continuous production of phenol from benzene and water without further oxidation of the product phenol will be possible. Further, the increased rate of the reactions, due to the large current flow through the plasma, is beneficial for this and other chemical reactions of interest.

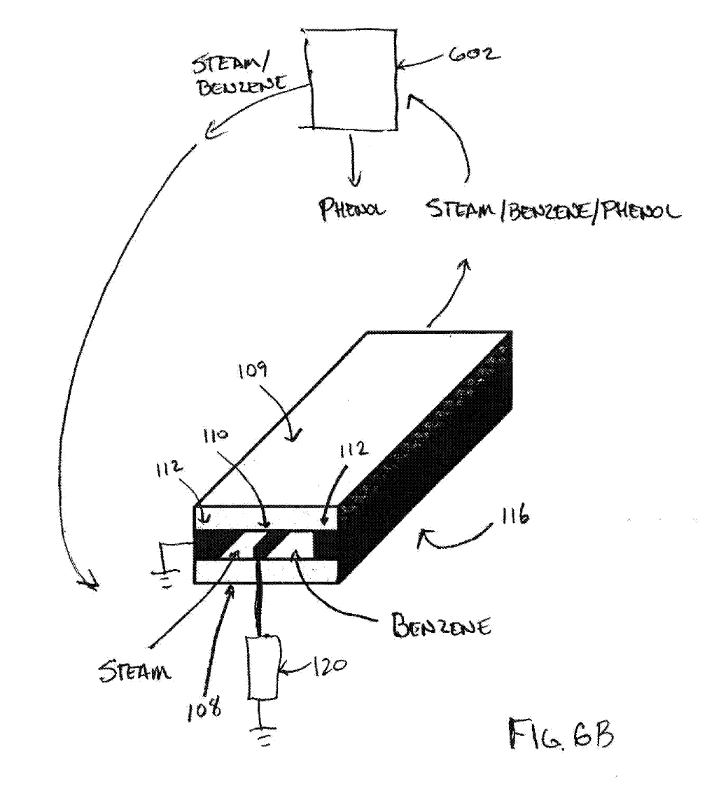

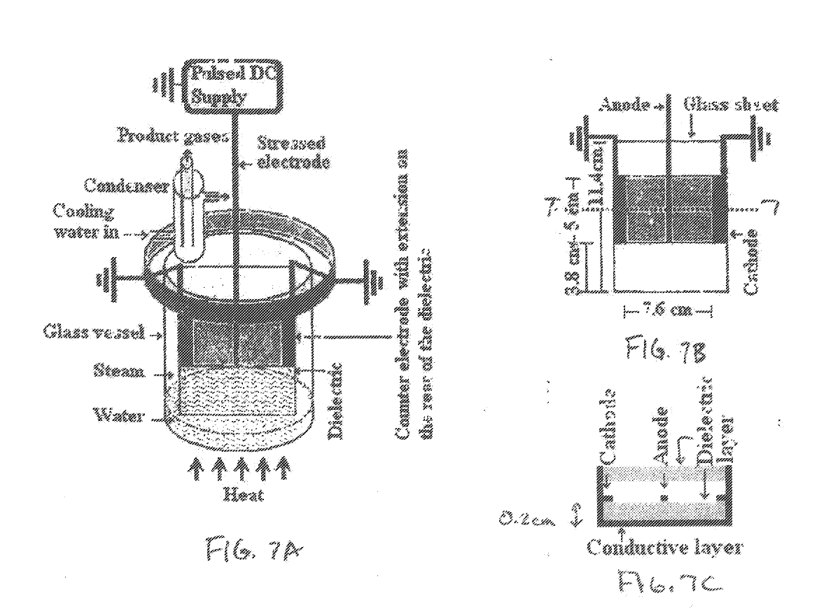

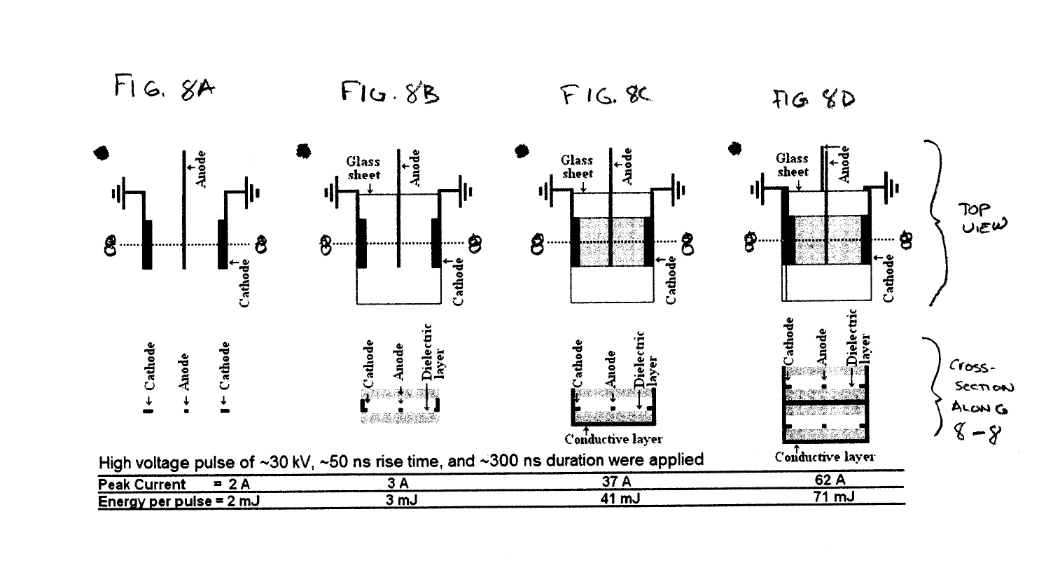

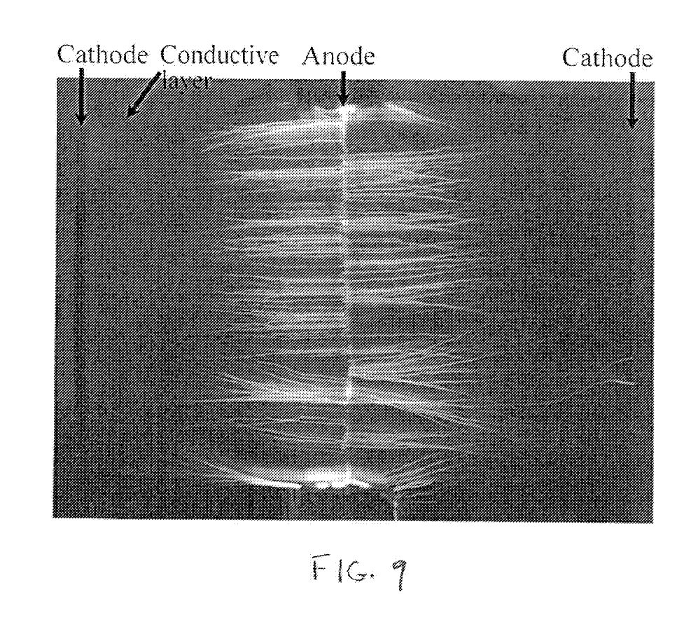

A schematic of an experimental setup of a reactor vessel in accordance with the various embodiments is illustrated in FIGS. 7A-7C. FIG. 7A is a schematic of the experimental setup. FIG. 7A shows an electrode assembly in the reaction vessel for the experimental setup with a power supply and a condenser connected. FIG. 7B shows a detailed view of the electrode assembly for the experimental setup. FIG. 7C shows a cross-section of the electrode assembly along cutline 7-7 in FIG. 7B.

For the experimental setup, steam was produced by boiling water in the reaction vessel to displace air from the reaction vessel. The water vapors were condensed in the condenser and recycled. The product gases formed as a result of the action of the plasma on the water molecules passed through the condenser. Flow rates of the product gases were measured by bubble flow meter and the composition of the product gases was analyzed by gas chromatography.

Three configurations of electrodes and dielectric were utilized to evaluate the electrode assembly described above. The first configuration uses a conventional pulsed corona configuration that discharges in steam (i.e., plasma is not in contact with dielectric surface). This configuration is illustrated in FIG. 8A, where the upper portion shows a schematic view of the electrodes for this configuration and the bottom portion shows a cross-section view through cutline 8-8. The second configuration uses plasma in contact with dielectric surface, but with no conductive layer on a second major surface. This configuration is illustrated in FIG. 8B, where the upper portion shows a schematic view of the electrode assembly for this configuration and the bottom portion shows a cross-section view through cutline 8-8. The third configuration, as shown in FIG. 8C, uses plasma in contact with dielectric surface with a conductive layer on the opposite side of the dielectric surface (the proposed reactor), similar to the configuration discussed above with respect to FIGS. 1A and 1B. The forth configuration, as shown in FIG. 8D, uses stacked discharge chambers. That is a stacking of two of the discharge chambers of FIG. 8C, as discussed above with respect to FIGS. 4A and 4B.

In the experimental setup, the applied voltage was 30 kV, the voltage rise time (10% to 90%) was 50 ns, pulse duration was 300 ns, and pulse repetition rate was 250 Hz. The peak current and electrical power were 2 A and 0.50 W in the case of plasma in steam in the absence of dielectric (FIG. 8A), 3 A and 0.75 W in the case of plasma in the presence of a dielectric (FIG. 8B), 37 A and 10 W in the case of plasma in contact with dielectric with a conductive layer on the opposite side of the dielectric (FIG. 8C), and 62 A and 18 W in the case of two stacked reactors (FIG. 8D), respectively.

The results described in the previous paragraph clearly show that both the current going through the plasma and the electric power increase by an order of magnitude in the proposed reactor (FIG. 8C) and it is scalable by stacking discharge chambers (FIG. 8D) as compared to the known non-equilibrium plasma reactors (FIG. 8A and FIG. 8B). The plasma was visible only in the proposed reactor (FIG. 8C). The resulting plasma is shown in FIG. 9. FIG. 9 is a time integrated image of atmospheric pressure non-equilibrium plasma in steam in the case of the electrode assembly of FIG. 8C.

A second experimental setup was employed to evaluate the performance of using multiple reactors in accordance with the various embodiments in parallel. This is shown in FIGS. 10A and 10B. FIG. 10A is an x-y plot of the production of hydrogen and oxygen with a single reactor shown in FIG. 1A. FIG. 10B is an x-y plot of the production of hydrogen and oxygen with two reactors operating in parallel shown in FIGS. 4A and 4B. Each of the reactors was configured as shown in FIGS. 7A-7D. Pure water vapor at 100° C. and one atmospheric pressure was treated by the plasma. The applied voltage was 30 kV and varying the pulse repetition rate varied the power. The rates of production are expressed in standard cubic centimeter per minute (sccm) at 0° C. and one atmospheric pressure and the energy yield is in grams of hydrogen produced per kilowatt hours (g H₂/kWh). Similarly, a continued increase in concentration of phenol with increase in treatment time was observed when a mixture of water and benzene were steam distilled to form vapors in the plasma zone of the reactor. The phenol concentration was qualitatively analyzed by UV-spectroscopy.

The energy efficiency in FIGS. 10A and 10B was estimated on the basis of rates of hydrogen produced by the dissociation of water molecules. Increasing the number of reactors from one to two increased the power and the rates of production of hydrogen and oxygen proportionately. The energy yield for production of hydrogen remained constant at 1.2 g H₂/kWh, showing the energy efficiency was constant in these conditions. These results also show that the power of the reactor can be increased by increasing the pulse repetition rate. Further, this also shows that it is possible to provide scaling of a reactor by operating multiple reactors in parallel.

The parameters related to the hydrogen production in the case of a system in accordance with the various embodiments are compared in Table 1 to the case of non-equilibrium plasma reactors employed by other research groups in the past. It can be observed from the 7th column in Table 1 that the energy yield in the present study (row number 6) is significantly higher than reported in the case of other reactors (row numbers 1 to 5). The value closest to that of the present study is that shown in row number 5, where a combined system of plasma and catalyst was employed. However, as noted above, hydrogen was generated in the absence of any catalyst in the reactor. Nonetheless, the use of a catalyst is possible in the various embodiments, which can further enhance the rate of hydrogen production.

TABLE 1
Comparison of hydrogen generation by dissociation of water molecules under the
action of atmospheric pressure non-equilibrium plasmas.

| | | | | | Rate of H₂ | |
|---|---|---|---|---|---|---|
| | | | | Temp.^{d} | Generation | Energy Yield |
| S. No. | Working Gas^{a} | Reactor^{b} | Power (W) | (° C.) | (mol/s) | (g H₂/kWh) |
| 1 | 1% H₂O_{(g)} | DBD | — | 25 | 0.37 × 10⁻⁸ | 0.0016 |
| | in nitrogen | | | | | |
| 2 | 1% H₂O | FPR | — | 25 | 24 × 10⁻⁸ | 0.051 |
| | in argon | | | | | |
| 3 | 2% H₂O_{(g)} | FPR | 25 | 25 | 41 × 10⁻⁸ | 0.12 |
| | in argon | | | | | |
| 4 | H₂O₍ₗ₎ | PCD | 37 | 25 | 130 × 10⁻⁸ | 0.25 |
| 5 | 2.3% H₂O_{(g)} | DBD^{c} | 0.94 | 25 | 8.5 × 10⁻⁸ | 0.65 |
| | in argon | | | | | |
| 6 | H₂O_{(g)} | SD | 33 | 100 | 530 × 10⁻⁸ | 1.2 |
| 7 | H₂O_{(g)} | MHC | 0.2 | 150 | 3.9 × 10⁻⁸ | 1.4 |
| 8 | H₂O_{(g)} | MIPC | 0.2 | 150 | 14 × 10⁻⁸ | 4.9 |
| 9 | H₂O_{(g)} | MIPC | 0.2 | 700 | 41 × 10⁻⁸ | 15 |

^{a}The subscript ‘g’ represents water vapor and ‘l’ represents liquid water;
^{b}DBD is dielectric barrier discharge reactor, FPR is ferroelectric pellet packed bed reactor, PCD is pulsed corona discharges, SD is sliding discharges on dielectric layer with a conductive layer on the opposite side of the dielectric (proposed reactor), MHC is micro-hollow cathode discharges, MJPC is micro-discharges in porous ceramic;
^{c}The plasma + catalysis by electrodes made of gold was employed to enhance the rate of hydrogen production;
^{d}25° C. is mentioned when the initial temperature of operation was reported to be room temperature.

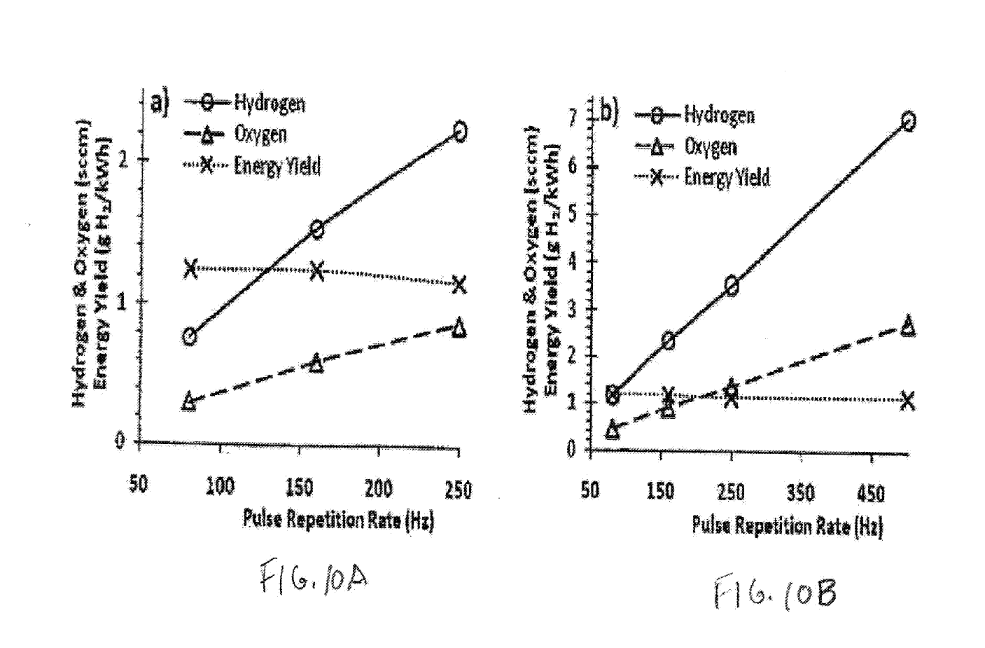

The energy yields in the case of row number 7, 8, and 9 are higher than that in the proposed reactor. The plasma in the case of reactors of row number 7, 8, and 9 was formed in tiny holes going through a dielectric layer. Such plasma through holes in a dielectric inherently has low power. This can be verified by the low power values and, consequently, low rates of hydrogen generation as shown in 4^{th} and 6th columns, respectively, in Table 1. In general, plasma in holes through a dielectric or in dielectric tubes is difficult to scale up. As previously noted, this is because the plasma in neighboring discharge gaps becomes coupled with adverse effects on each other. The plasma leaves charges on the dielectric surface, which induces the charges of opposite polarity on the other side of the dielectric. The induced charges have adverse effects on the plasma formation in the neighboring chambers when they are operated in parallel. This is discussed below with respect to FIGS. 11A-11C.

FIGS. 11A-11C schematically illustrate the experimental setup for showing the adverse effects on the plasma formation in the neighboring chambers when they are operated in parallel, but without conductive layers between cylindrical dielectrics. In particular, FIGS. 11A, 11B, and 11C show schematically the experimental setup for one, two, and three surface plasmas, respectively. No increase in power upon operating multiple plasma reactors in such dielectric tubes was observed. For these experiments, a glass tube was used with a 6 mm inner diameter, 7 mm outer diameter and 90 mm length was employed. The stressed electrode was a loop of stainless steel wire of 150 um diameter placed in the dielectric tube and electrically connected to the pulsed power supply. The counter electrode was made of aluminum foil of 150 um thickness placed at the end of the dielectric tube. The inter-electrode gap of 50 mm was filled with air. In the case of the single tube (FIG. 11A), peak current was 0.83 A and power was 2.2 mW when pulses of 30 kV were applied at the rate of 4 Hz. The peak current and power decreased to 0.74 A and 1.8 mW in the case of two tubes (FIG. 11B) and further decreased to 0.064 A and 1.5 mW, respectively, in the case of three tubes (FIG. 11C) operated in parallel under the same experimental conditions. Similar results were observed in the case of the discharge using an electrode assembly in accordance with the various embodiments. In such a configuration, the power decreased from 1.1 W in a single reactor to 0.8 W in two reactors operated in parallel when the working gas was air, the effective length of the electrodes was 130 mm, and pulses of 30 kV were applied at the rate of 500 Hz.

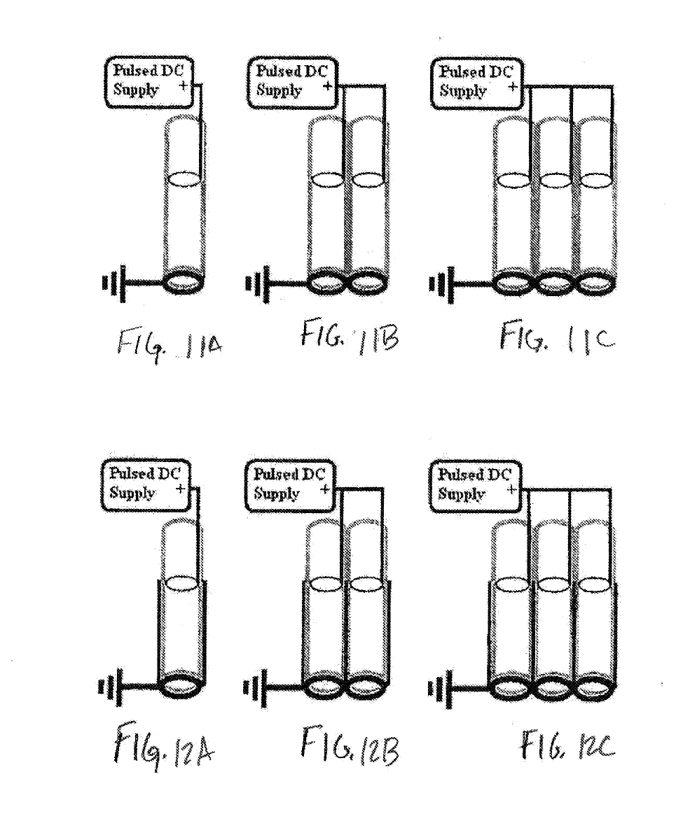

The effect of shield portions separating the dielectric tubes on increasing power and scalability by operating multiple plasma reactors in parallel is demonstrated by employing the setup shown in FIGS. 12A-12C. FIGS. 12A-12C schematically illustrate the experimental setup showing the effects on the plasma formation in the neighboring chambers when they are operated in parallel and are separated by shield portions. In particular, FIGS. 12A, 12B, and 12C show schematically the experimental setup for one, two, and three surface plasmas, respectively. In the case of a single tube, the peak current and power, which were 0.83 A and 2.2 mW in the absence of the shield portion increased to 25 A and 138 mW in the presence of a shield portion consisting of a conductive layer or film wrapped around the tube (FIG. 12A) under the same experimental conditions. This is about an order of magnitude increase in the electrical power. The peak current and electrical power increased to 44 A and 218 mW in the case of two tubes (FIG. 12B) and further increased to 67 A and 315 mW, in the case of three tubes (FIG. 12C) operated in parallel in the presence of the conductive layer under the same experimental conditions. In the experimental setup, aluminum foil was used to provide the shield portions. Further the shield portions were configured to be extensions of the grounded electrodes, i.e., also grounded. Similar results were observed in the case of the discharge using an electrode assembly in accordance with the various embodiments. In this configurations, the combination of the electrode assembly shown in FIG. 8C and the shield portions results in the power increasing from 1.3 W in single reactor to 2.0 W in two reactors operated in parallel when working gas was air, using effective length of the electrodes equal to 130 mm, and pulses of 30 kV applied at the rate of 20 Hz.

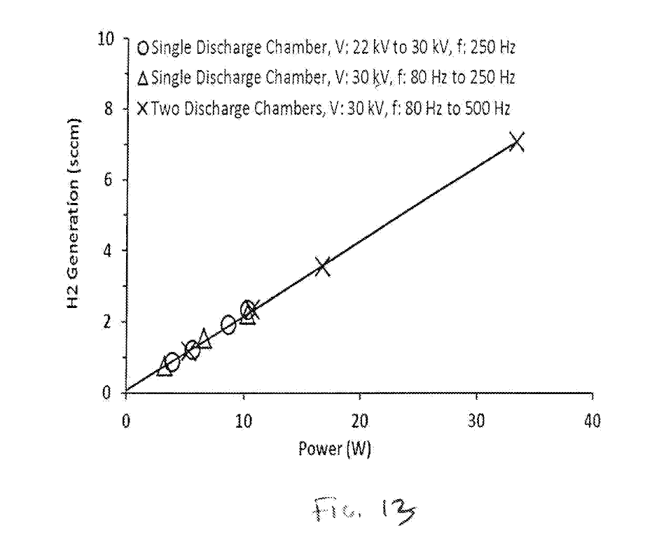

The advantage of multiple chambers is further illustrated in FIG. 13. FIG. 13 is a plot of hydrogen generation as a function of power. The first set of data (circles) shows the results for a single discharge chamber for voltage pulses between 22 kV and 30 kV at a frequency of 250 Hz. The second set of data (triangles) shows the results for a single discharge chamber for voltage pulses of 30 kV at frequencies between 80 Hz and 250 Hz. The third set of data (hatches) shows the results for two discharge chambers for voltage pulses of 30 kV at frequencies between 80 Hz and 500 Hz. As shown in FIG. 13, when a single chamber is used, the results are essentially the same, regardless of whether voltage is varied (circles) or whether frequency of pulses is varied (triangles). However, when the two discharge chambers are used, the total power that can be delivered is significantly increased, resulting in significantly higher hydrogen generation as compared to a single discharge chamber.

The improved efficiency of the various embodiments is readily ascertainable when compared to other comparable treatment systems. This is shown in Table 2 (row 6 is a reactor in accordance with the various embodiments):

TABLE 2
Values of parameters related to hydrogen generation from
water molecules by the action of non-equilibrium plasmas.

| | | | | | Rate of H₂ | H₂ | Energy |
|---|---|---|---|---|---|---|---|
| S. | | | Power | Temperature | Generation | Concentration | Yield |
| No. | Working Gas | Reactor^{a} | (W) | (° C.) | (μmoles/s) | (%) | (g H₂/kWh) |
| 1 | 1% H₂O_{(g)} in nitrogen | DBD | — | 23^{d} | 0.0037 | 0.005 | 0.0016 |
| 2 | 1% H₂O_{(g)} in argon | FPR | — | 23^{d} | 0.24 | 0.3 | 0.051 |
| 3 | 2% H₂O_{(g)} in argon | FPR | 25 | 23^{d} | 0.41 | 0.04 | 0.12 |
| 4 | H₂O₍ₗ₎ | PCD^{b} | 37 | 23^{d} | 1.3 | 0.4 | 0.25 |
| 5 | 2.3% H₂O_{(g)} in argon | DBD^{c} | 0.94 | — | 0.085 | 0.2 | 0.65 |
| 6 | H₂O_{(g)} | SD | 33 | 100 | 5.3 | 60 | 1.2 |
| 7 | H₂O_{(g)} | MHC | 0.2 | 150 | 0.039 | 0.3 | 1.4 |
| 8 | H₂O_{(g)} | MICP | 0.2 | 150 | 0.14 | 0.3 | 4.9 |
| 9 | H₂O₍ₗ₎ spray in argon | GA | 0.3 | 23^{d} | 0.55 | 0.04 | 13 |
| 10 | H₂O_{(g)} | MICP | 0.2 | 700 | 0.41 | 0.9 | 15 |

^{a}FPR is ferroelectric pellet packed bed reactor, DBD is dielectric barrier discharge or silent discharge reactor, PCD is pulsed corona discharges, SD is sliding discharges on dielectric with a conductive layer on the opposite side of the dielectric (proposed reactor), MHC is micro-hollow cathode discharges, GA is GlidArc, and MICP is micro-discharges in porous ceramics,
^{b}gas was bubbled through the liquid water during the plasma operation,
^{c}the effects of the plasma were combined with catalysis by the electrode made of gold, and
^{d}temperature in the reactor is not known but the feed mixture was at room temperature. As shown in the table above, significantly higher rates of hydrogen generation, higher hydrogen concentrations, and higher energy yields were obtained using a reactor in accordance with the various embodiments.

In some embodiments, the materials and thicknesses thereof can affect the amount of species generated. For example, in the case of steam, the amount of hydrogen and hydrogen peroxide can be affected by the type of dielectric material for the dielectric sheets and the thicknesses thereof. Various materials and thicknesses thereof were evaluated, as shown in Table 3:

TABLE 3
Material, Thickness, and Electrical Characteristics

| | | Thickness of | Peak | Energy per |
|---|---|---|---|---|
| S. No. | Dielectric | dielectric (cm) | current (A) | pulse (mJ) |
| 1 | Glass | 0.32 | 22 | 21 |
| 2 | Glass | 0.48 | 14 | 16 |
| 3 | Macor | 0.32 | 13 | 15 |
| 4 | Macor | 0.48 | 11 | 11 |

For each of these cases, peak voltage ˜30 kV, rise time of the voltage (10% to 90%) ˜50 ns and pulse width at half maximum ˜150 ns. The results for these cases are shown in FIGS. 23-26.

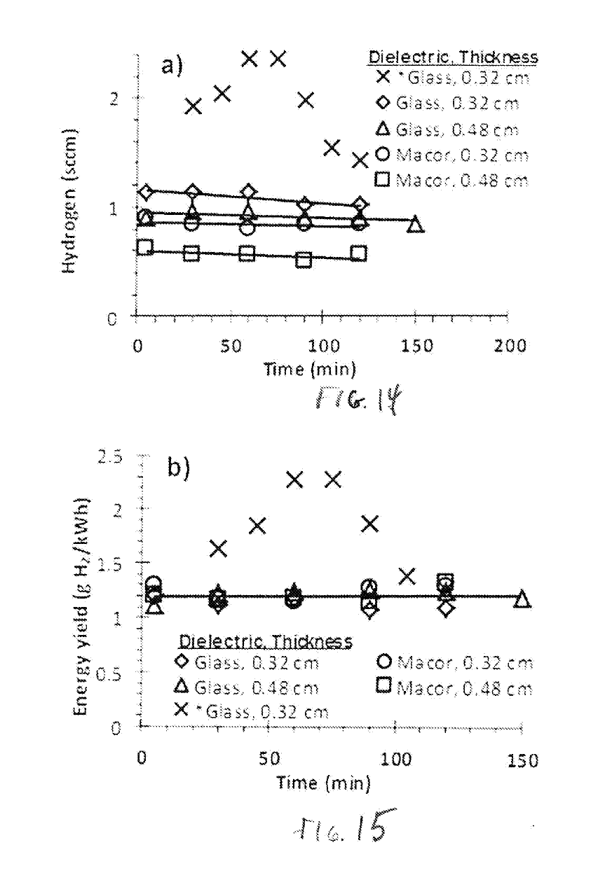

FIG. 14 is a plot of hydrogen generation as a function of time and material for steam as a working gas. As shown in FIG. 14, glass provides a greater hydrogen generation rate than MACOR, a machinable glass-ceramic developed and sold by Corning Inc. of Corning, N.Y. Further, for both MACOR and glass, as thickness is increased, hydrogen generation rates decrease. FIG. 15 is a plot of energy yield as a function of time for the materials in FIG. 14. As shown in FIG. 15, the type of material and the thickness does not appear to have a significant effect on energy yield. In FIGS. 14-19, the hash marks (“X”) indicate the effect of the addition of Benzene to the steam.

FIG. 14 shows that with steam as a working gas, the rate of hydrogen generation decreases with increasing thickness of the dielectric layer, and it also decreases when the glass dielectric is replaced by Macor of a same or similar thickness. This trend follows the changes in energy deposition shown in Table 3. However, the energy yield for hydrogen generation is 1.2±0.1 g/kWh independent from input power, and thickness or material of the dielectric as shown in FIG. 15. The percentage of hydrogen was 73±4% and that of oxygen 18±1% in the gaseous products.

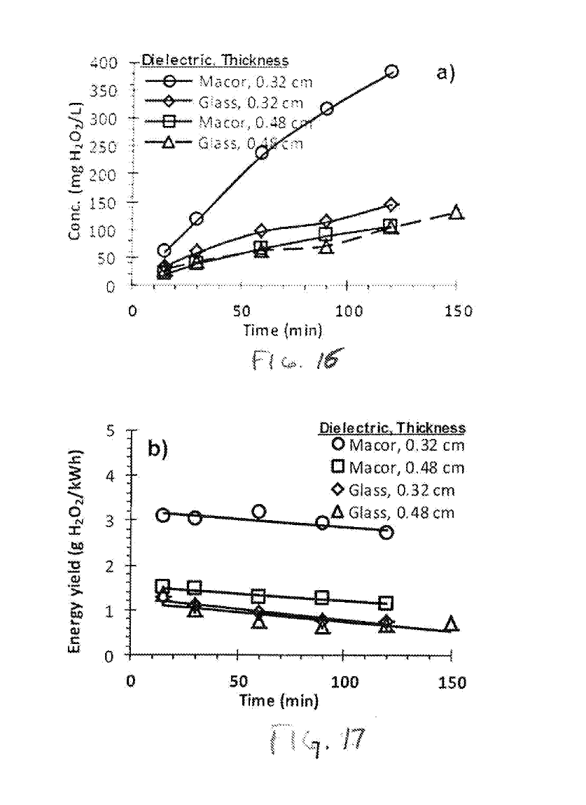

FIG. 16 shows that hydrogen peroxide was formed and accumulated in the condensed liquid. The highest rate of formation and the highest energy yield was in the case of Macor with 0.32 cm thickness. There was a significant decrease in the rate and energy for hydrogen peroxide when the thickness of Macor was increased to 0.48 cm. The rate and energy yield were significantly lower with window glass, as shown in FIG. 17. Furthermore, for window glass as dielectric the energy yield was independent of thickness of dielectric.

FIG. 16 is a plot of hydrogen peroxide generation as a function of time and material. As shown in FIG. 16, MACOR provides a greater hydrogen peroxide generation rate than glass. Further, for both MACOR and glass, as thickness is increased, hydrogen peroxide generation rates decrease. FIG. 17 is a plot of energy yield for hydrogen peroxide as a function of time for the materials in FIG. 16. As shown in FIG. 17, the type of material appears to have a significant effect on energy yield for hydrogen peroxide. Specifically thinner MACOR appears to provide substantially higher energy yields.

In summary, the foregoing datasets show that the energy deposition in the plasma can be increased by increasing the capacitance of the plasma-dielectric-conductive layer. This can be achieved by: (1) reducing the thickness of the dielectric layer; or (2) increasing the permittivity of the dielectric (this has been shown by using Macor and glass, with glass having a higher permittivity than Macor). The energy yield for hydrogen is not affected by variations in input power, treatment time and thickness or material of the dielectric. The energy yield for hydrogen peroxide is dependent on material of the dielectric—higher for Macor than for glass. In the case of Macor it is dependent on the thickness of the dielectric indicating a major role of surface mediated reactions which lead to the generation of hydrogen peroxide.

Additionally, treatment time can have an effect as well. FIG. 18 is a plot of absorbance data reflecting Benzene to Phenol conversion in steam plasma for different treatment times versus and a control Phenol sample. The inset plot in FIG. 18 shows the calibration curve for absorbance and Phenol concentration. In FIG. 18, the Phenol concentration can be evaluated from the peak absorbance data using the calibration curve. FIG. 18 shows that as the amount of treatment time is increased, the peak absorbance is also increased, resulting in increased Phenol generation. For example, a 30 min treatment will result in a less than 1 mg/L, while a 120 min treatment will result in approximately 3 mg/L.

In addition to the foregoing variables, the polarity can affect efficiency. This is shown in FIG. 19. FIG. 19 is a plot of NO removal as a function of input energy for various gaps, lengths, and pulses, including pulses of different polarities. As can be observed from FIG. 19, negative polarity pulses (anode directed, circles) are clearly more efficient for NO removal than positive polarity (cathode directed, squares). This can be related with the longer length of the anode directed streamers and lower applied voltage. However, cathode directed streams can also be configured to provide greater efficiencies at lower applied voltages. For example, the cathode directed streams with 8 kV voltage and 5 mm inter-electrode gap (hatches) are clearly the most efficient in FIG. 19. This is because at lower applied voltage each individual streamer carries less energy which is beneficial for utilization of the reactive species in the desired chemical reaction, but the effective length of electrode was increased which increases their number. This spatial distribution of the streamers makes them more efficient without much reduction in energy going into the plasma for chemical reactions.

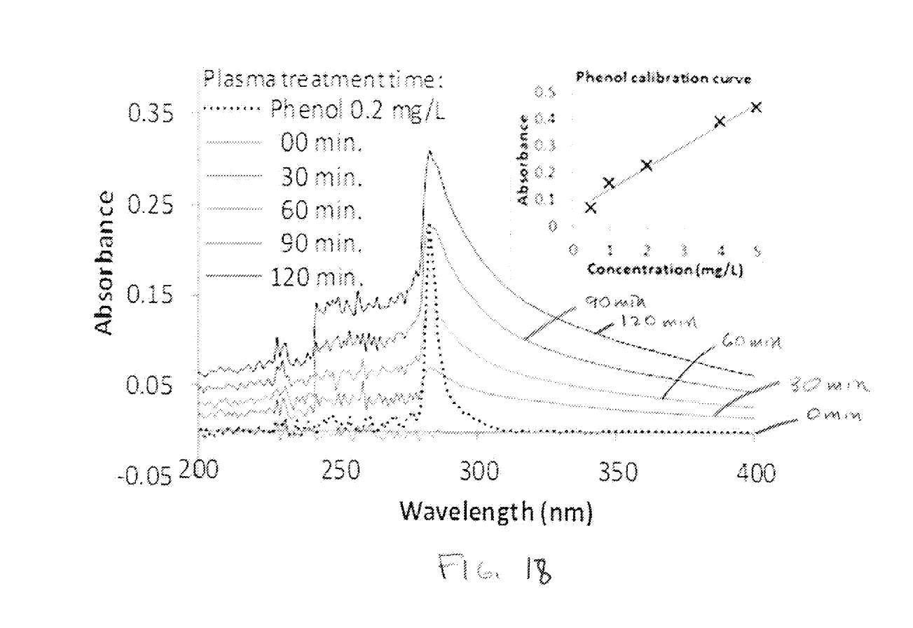

In the foregoing discussion, the various embodiments have been described as including electrodes positioned such that the streamers propagate in a direction relatively perpendicular to the direction of the gas flow. In particular, the electrode are substantially parallel to the direction of gas flow, as shown in FIGS. 1A-1B and 4A-4B. However, the various embodiments are not limited in this regard. That is, in other embodiments, the electrodes can be arranged such that the streamers propagate in a direction relatively parallel to the gas flow. Such a configuration is illustrated in FIG. 20.

FIG. 20 shows a treatment system 2000 in accordance with another embodiment of the invention. Similar to the configuration in FIGS. 1A-1B, the system 2000 includes opposing dielectric or glass sheets 2004 defining a discharge space 2002 plasma channels form and propagate, an anode electrode 2010, and cathode electrodes 2012. As previously noted, the designations “anode” and “cathode” are provided solely for illustrative purposes and the function of electrodes 2010 and 2012 can be interchanged.

System 2000 can be operated in substantially a same manner as system 100 in FIGS. 1A-1B. Significantly, electrode 2010 and electrodes 2012 are positioned at opposite ends of the discharge space 2002, corresponding to inlet portion 2014 and outlet portion 2016, respectively. As a result, during operation of system 2000, the streamers formed therein propagate in substantially a same direction as the gas flow. Similar to the system in FIGS. 1A-1B, multiple ones of system 2000 can be combined to provide scaling as previously described.

In some configurations, the system 2000 can include a slit cover 2018 with at least one slit. The slit can be between 0.01 cm and 10 cm in length and between 0.01 cm and 1 cm in width. Further, multiple slits can be provided in the slit cover 2018. Alternatively, the glass sheets can be spaced apart to provide a spacing between 0.01 cm and 1 cm and thus define one or more slits.

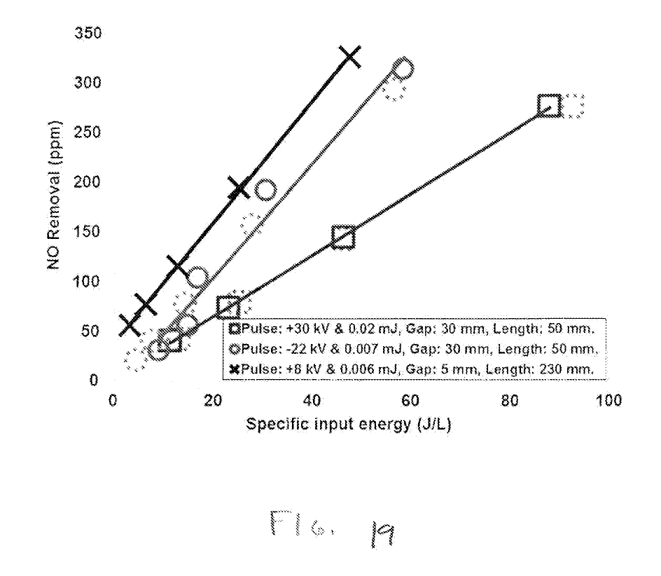

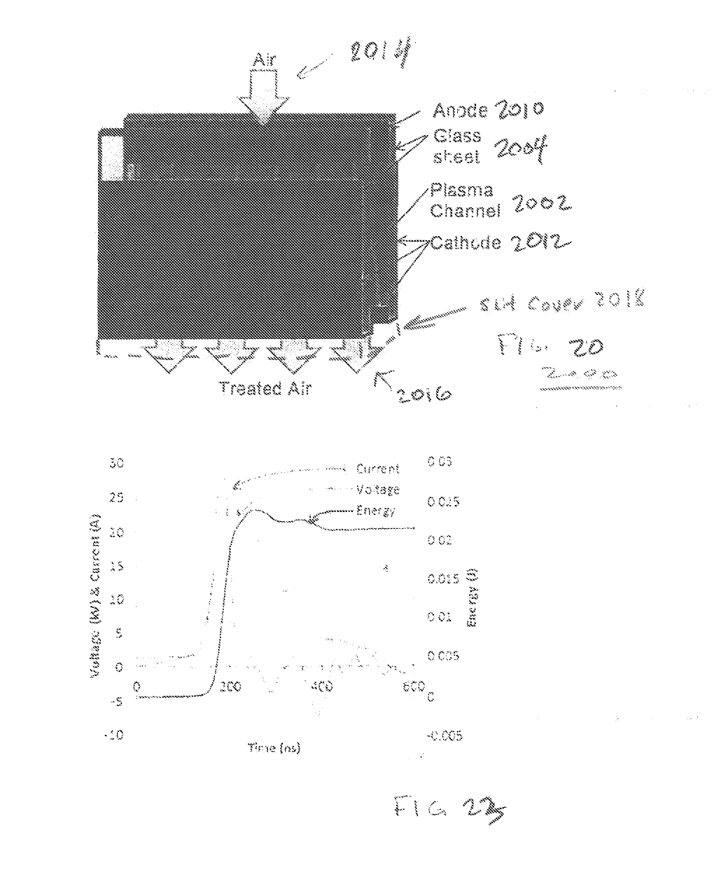

The resulting streamers for system 2000 are shown in FIGS. 21A and 21B and FIGS. 22A and 22B. FIGS. 21A and 21B are Images of plasma in large slit and small slit, respectively. The conductive layer on top has been removed to view the plasma in the discharge gap. However, the conductive layer on bottom, i.e, opposite to electrodes in the discharge chamber was still in place. Further, as noted above, the streamers are not significantly affected due to electrode orientation. FIGS. 22A and 22B show cathode directed streamers and anode directed streamers (i.e., with reverse polarity) respectively.

Despite the change in orientation of the streamers, no adverse effects on gas treatment are observed. Rather, a plasma reactor assembled using the system 2000 results in an increase in electrical energy deposition in non-thermal plasma by an order of magnitude compared to conventional cold plasmas. Rates of production of chemically active species and the chemical reactions driven by them were found to increase in proportion to the energy deposition in the plasma. Thus, such a reactor design not only allows generation of an energetic plasma in air, it can also be used to generate a scalable and energetic plasma in the presence any gas, e.g. in air and water vapor mixtures of any proportions. In one example, water vapor in a process gas can be used for treatments. Such a configuration is advantageous as the formation of strong antibacterial agents like hydroxyl radicals and hydrogen peroxide, generated in water vapor plasma, makes this non-thermal plasma ideally suited for bacterial decontamination.

In the various embodiments, a system, such as that of system 2000 or any other system described herein, can allow plasma-activated gas to exit through a slit, as illustrated in FIGS. 21A and 21B and FIGS. 22A and 22B. In one example, the slits can be approximately one millimeter in width and a few decimeters in length. However, the various embodiments are not limited in this regard and other sizes of slits can be used. Further, multiple plasma slits can be operated in parallel, allowing the decontamination of large surface areas.

The current and voltage waveforms and the cumulative energy per pulse (with air as a working gas) for the system 2000 are shown in FIG. 23. The nanosecond pulses generate sliding streamer discharges along the glass in a discharge chamber of 5.4 cm×2.5 cm×0.16 cm (slit width). The plasma-activated gas (either air or air with water vapor or their mixtures with other gases) exits at the bottom through a slit which is 5.4 cm×0.16 cm. In the case of bacterial decontamination, the voltage pulses were run at applied voltage of 25 kV, repetition rate of 500 Hz at a peak power of approximately 0.6 MW, and an average power level of only ˜10 W.

In order to determine the influence of the slit width or the distance between the dielectric layers (glass) in the discharge chamber, respectively, a device with a narrower slit was constructed and studied. In this case the cold plasma is generated in a discharge chamber of 2.6 cm×2.0 cm×0.029 cm. The plasma-activated gas (either air or air with water vapor) exits at the bottom through a slit which is 2.6 cm×0.029 cm. The appearance of the plasma formed in the smaller slit device is similar to that generated in the larger slit device. The average power of ˜5 W was lower than in the earlier device (˜10 W).

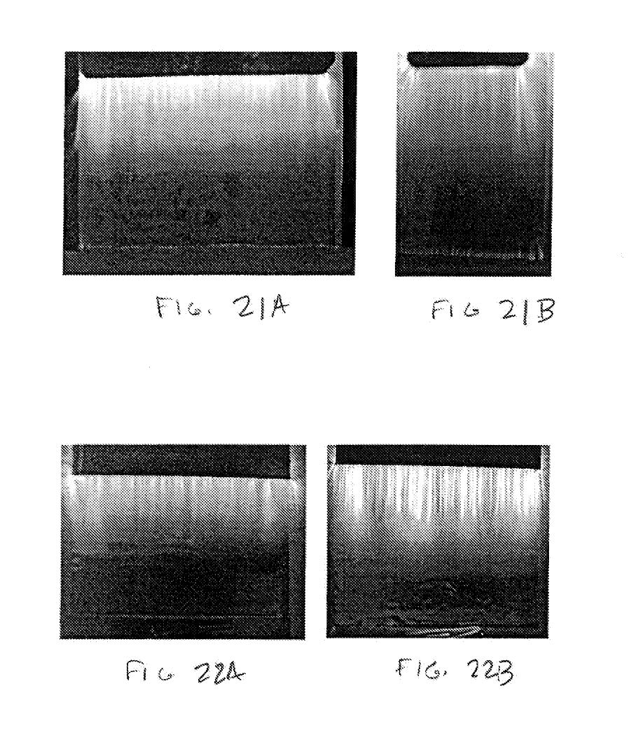

When the discharge was operated with positive high voltage pulses applied to the inner electrode (electrode 2010), cathode-directed streamers in the discharge gap were generated at an applied voltage of ˜25 kV. When the polarity was reversed, with the inner electrode is configured as the cathode, anode-directed streamers were generated at a lower applied voltage (˜22 kV) and with reduced energy consumption (˜2.5 W). This result indicates that a considerable reduction in voltage and energy can be achieved with discharges which generate anode-directed streamers, rather than cathode-directed streamers. Although the energy consumption was different for differently biased electrodes, the ozone concentration in the treated gas was not affected by the change in polarity. This is illustrated in FIG. 24. FIG. 24 shows a plot of ozone concentration (in ppm) as a function of flow rate of air for cathode and anode directed streamers.

Further, the gram-negative Escherichia coli and the gram-positive Staphylococcus epidermidis were used as model microorganisms for opportunistic pathogens in this study. Overnight nutrient-rich broth cultures of E. coli and S. epidermidis were serially diluted to a final concentration of 10⁴ cells/mL. 100 μL of each bacterial suspension was uniformly spread on Brain Heart Infusion agar plates. No polymicrobial cultures were used in these experiments; instead bacterial species were treated separately. Seeded plates were air-dried to insure proper adherence of cells to the solid medium.

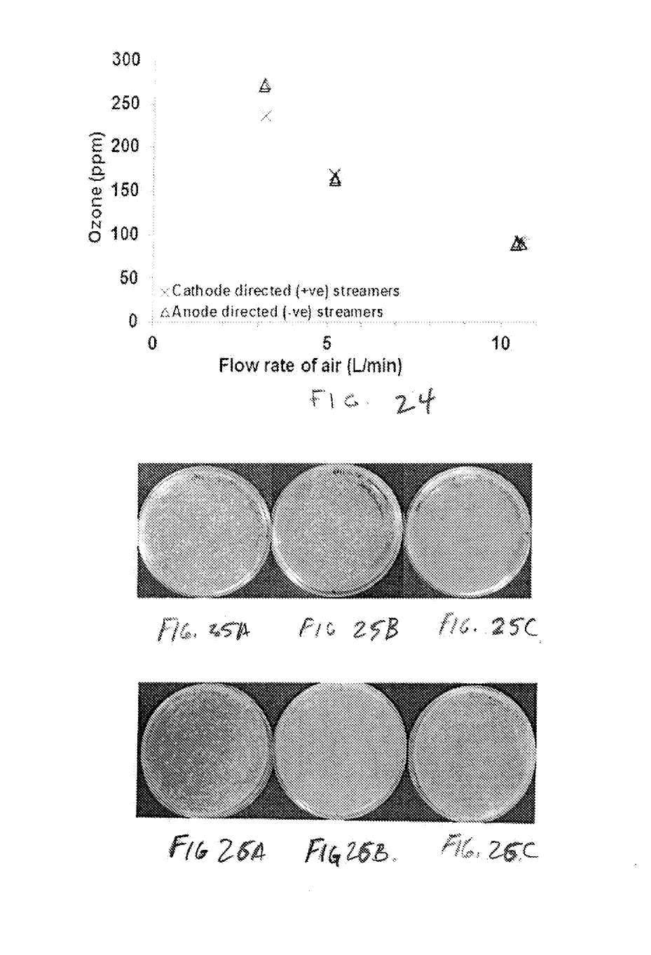

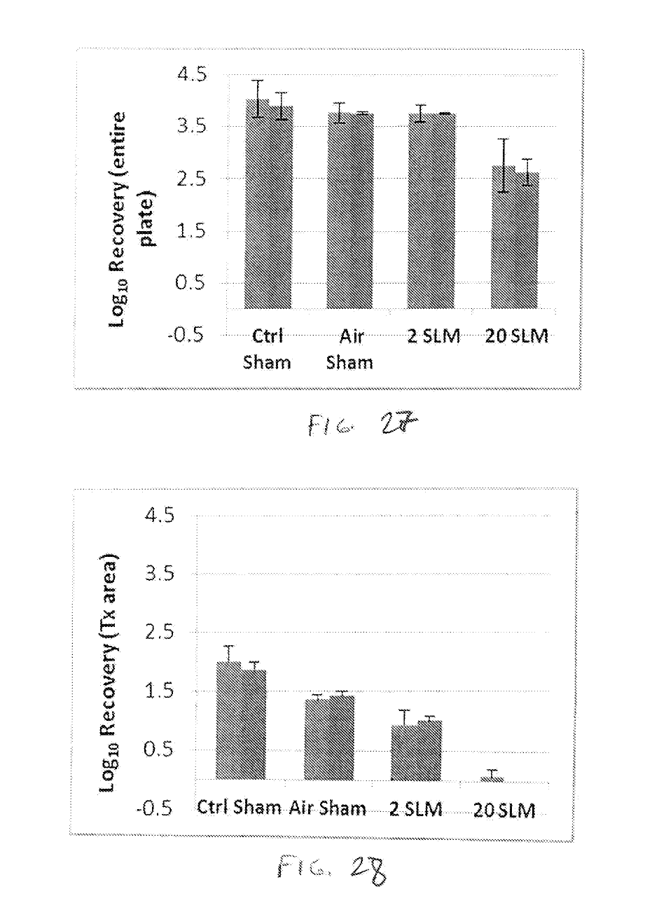

The plasma wand or slit was positioned laterally across the seeded petri dish at a constant distance of 10 mm from the agar's surface. This orientation divided the plate into two equal hemispheres, ensuring an approximately similar treatment necessary for establishing the boundaries of a potential bystander effect. The applied voltage was 25 kV, repetition rate was 250 Hz and the duration of treatment for these exposures was 3 minutes. Post-treatment, agar plates were incubated overnight at 28° C. and subsequent bacterial recovery determined compared to an untreated control. The result of such treatments are shown in FIGS. 25A-25C and 26A-26C.

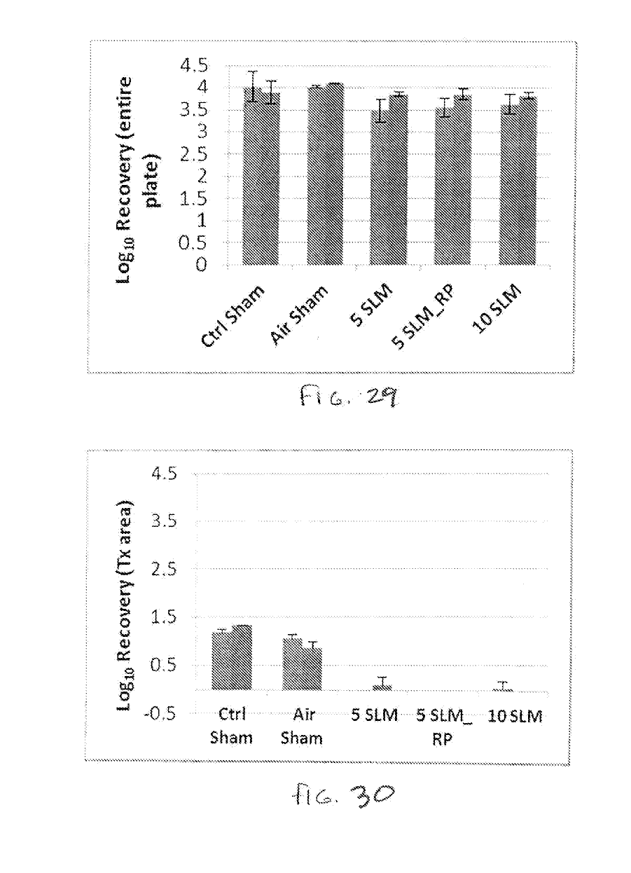

FIGS. 25A-25C show the results of the larger slit on S. epidermidis for Air sham (FIG. 25A), 2 standard litter per minute (SLM) air (FIG. 25B), and 20 SLM air (FIG. 25C). FIGS. 26A-26C show the results of the smaller slit on E. coli for Air sham (FIG. 26A), 5 SLM air (FIG. 26B), and 5 SLM air (FIG. 26C). The results suggest that the larger slit plasma wand device was highly effective against inactivating both E. coli and S. epidermidis in the direct treatment area at an operating flow rate of 20 standard litters per minutes (SLM), yielding 0.09±0.11 and 0.00±0.00 Log₁₀ recovery respectively when compared to 2.00±0.26 and 1.87±0.13 Log₁₀ recovery from the untreated controls (as shown in FIG. 28). The antibacterial effect is less pronounced when operated at 2 SLM. Overall, the same inactivation trend is observed when considering the Log₁₀ recovery on the entire plate, including both directly and indirectly treated areas (as shown in FIG. 27). This observation indicates the presence of a bystander effect achieved from stationary application of this device.

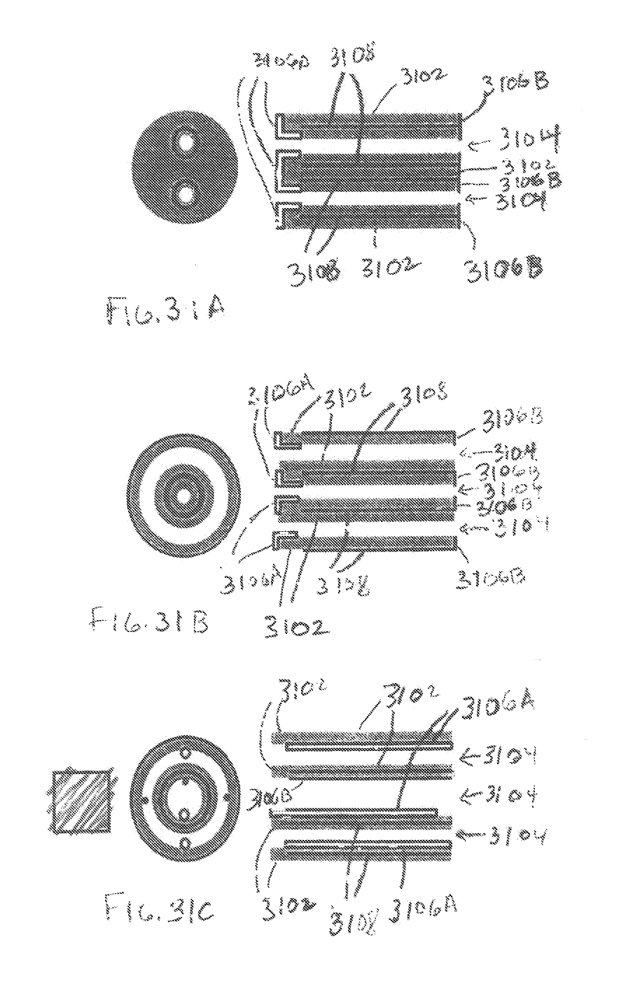

Results from the Smaller Slit plasma wand device suggest the smaller area opening to have greater efficacy when operated at lower flow rates than experimentally used for the Larger Slit device. At flow rates of 5 and 10 SLM, the Smaller Slit plasma wand reduced the seeded E. coli inoculum by roughly 0.5 Log₁₀ across the entire plate. Although slightly less effective, at these same operating flow rates the Smaller Slit plasma wand reduced the seeded S. epidermidis inoculum by approximately 0.25 Log₁₀ considering the whole plate (FIG. 29). Within the directly treated area, a flow rate of 5 SLM at normal (cathode directed streamers) and reverse (anode directed streamers) electrode polarity (RP) yielded a non-detectable E. coli growth recovery, while operation at 10 SLM resulted in a 0.05±0.12 Log₁₀ recovery (FIG. 30). Effectiveness against S. epidermidis was equally successful. At flow rates of 5 SLM, 5 SLM_RP, and 10 SLM the bacterial recoveries were 0.1±0.17 Log₁₀, 0.0±0.0 Log₁₀), and 0.0±0.0 Log₁₀ respectively (FIG. 30).

Both of these wand devices show efficacy against inactivation of surface contaminations of E. coli and S. epidermidis. We observed little killing differences between these two model gram-negative and gram-positive microorganisms, suggesting the possibility of broad-spectrum specificity when using this device; although the lack of pathogenicity of these bacteria is noted. The inclusion of the recovered growth across the entire plate demonstrates the need for multiple adjacent slits for the treatment of larger areas.

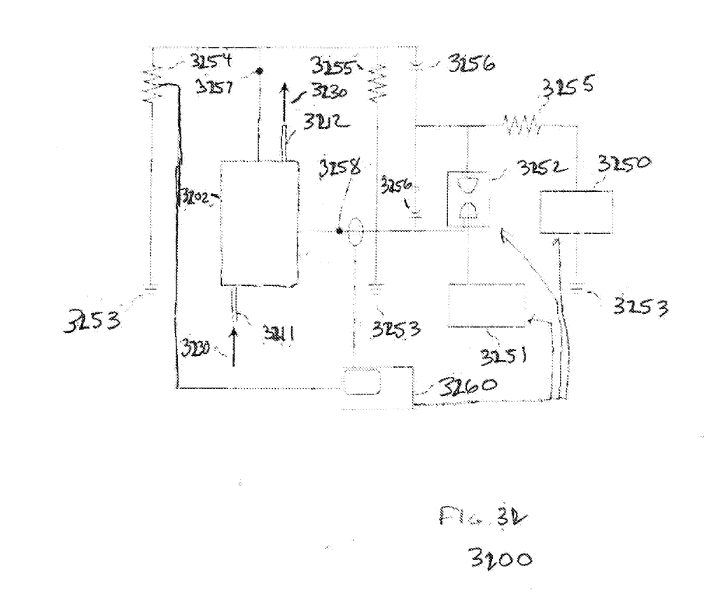

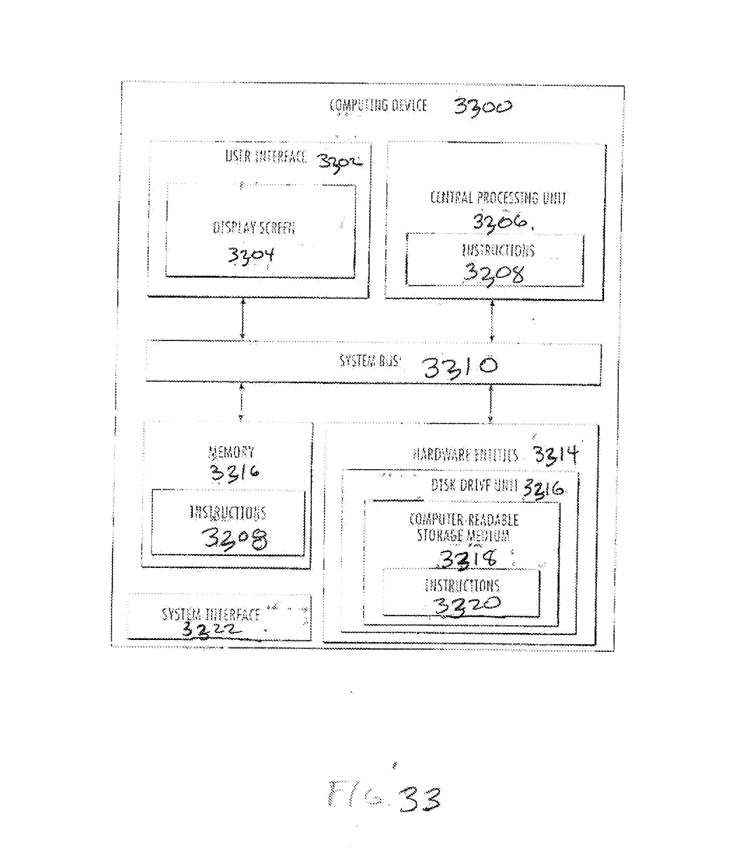

The configurations described above rely on the placement of a planar dielectric electrode device disposed in a cylindrical chamber, where the discharges are generated in the gas or vapor space between electrode and counter-electrode, along the planar dielectric. However, the various embodiments are not limited to solely the configurations described above. Rather, non-planar configurations can be used in the various embodiments. In particular, the electrodes can be positioned on the wall of the cylindrical chamber. Some examples of such alternate configurations are illustrated below with respect to FIGS. 31A-31C

FIG. 31A shows side, front cross section, and side cross-section views for a reactor geometry with multiple (only two are shown) cylindrical reactor chambers, consisting of two or more cylindrical layers of dielectric 3102 and conductors 3108 surrounding the discharge chambers in a dielectric tube. The discharges are generated in the gas or vapor space 3104 in the tubular discharge chambers between electrode 3106A and counter-electrode 3106B (in axial direction).

FIG. 31B shows side, front cross section, and side cross section views for a coaxial reactor geometry consist of concentric layers of dielectric 3102/conductor 3108/dielectric 3102 which separate the individual cylindrical reactor chambers. The discharges are generated in the gas or vapor space 3104 in the tubular discharge chambers between electrode 3106A and counter-electrode 3106B, which are located at either end of the cylinder (note: the counter-electrode 3106B can be connected to the conductive layer 3108 which, together with the dielectric tubes, separates the discharge chambers).

FIG. 31C shows another coaxial reactor geometry, however, with electrodes arranged such that the discharge is developing in azimuthal direction, rather than in longitudinal direction as in FIG. 31B. The electrodes 3106A and counter-electrodes 3106B in this case consist of wires on top of the dielectrics 3102 in longitudinal (axial) direction, causing the discharge to develop along the perimeter of the cylindrical, coaxial dielectric tubes, rather than in axial direction. Also, in order to keep the discharge gap (distance between electrode 3106A and counter electrode 3106B) equidistant, a requirement if pulses of the same duration are used for each discharge chamber, multiple electrode arrangements in azimuthal directions have to be used. In the example shown in FIG. 31C with only two discharge chambers, the inner discharge chamber has only a single electrode and counter-electrode, whereas the chamber with a larger radius, has two electrode systems, where the distance between the various electrodes is about the same as in the innermost discharge chamber.

Although in these examples we have shown reactor assemblies with a small number of individual discharge chambers, it is obvious that these geometries can be expanded to include multiple, stacked discharge chambers.

FIG. 32 illustrates a system including a gas treatment device 3202, configured in accordance with an embodiment of the invention, and supporting electrical circuitry. In operation, a high voltage pulse can be applied to device 3202. In the various embodiments, the pulse can be formed using an L-C inversion circuit, with trigger generator 3251, spark gap switch 3252, resistor 3255, capacitors 3256, and high voltage direct current power supply 3250. This pulse was applied to high voltage electrode node 3257 (i.e., the anode electrode), while counter electrode node 3258 (i.e., the cathode electrode and/or shield portions) was grounded (i.e., coupled to ground node 3253). A control system 3260 can be provided to monitor and control the various elements in system 3200. Other components can also be provided, such as resistor 3254 for providing a voltage divider for measuring voltage. The pulse duration preferably is short enough to prevent the occurrence of a transition from streamer to arc. Those skilled in the art will readily see that a variety of circuits may be used and pulses having different characteristics may readily be achieved.

Referring now to FIG. 33, there is provided a detailed block diagram of a computing device 3300 which can be implemented as control system 3260. Although various components are shown in FIG. 33, the computing device 3300 may include more or less components than those shown in FIG. 33. However, the components shown are sufficient to disclose an illustrative embodiment of the invention. The hardware architecture of FIG. 33 represents only one embodiment of a representative computing device for controlling a jointed mechanical device.

As shown in FIG. 33, computing device 3300 includes a system interface 3322, a Central Processing Unit (CPU) 3306, a system bus 3310, a memory 3316 connected to and accessible by other portions of computing device 3300 through system bus 3310, and hardware entities 3314 connected to system bus 3310. At least some of the hardware entities 3314 perform actions involving access to and use of memory 3316, which may be any type of volatile or non-volatile memory devices. Such memory can include, for example, magnetic, optical, or semiconductor based memory devices. However the various embodiments of the invention are not limited in this regard.

In some embodiments, computing system can include a user interface 3302. User interface 3302 can be an internal or external component of computing device 3300. User interface 3302 can include input devices, output devices, and software routines configured to allow a user to interact with and control software applications installed on the computing device 3300. Such input and output devices include, but are not limited to, a display screen 3304, a speaker (not shown), a keypad (not shown), a directional pad (not shown), a directional knob (not shown), and a microphone (not shown). As such, user interface 3302 can facilitate a user-software interaction for launching software development applications and other types of applications installed on the computing device 3300.

System interface 3322 allows the computing device 3300 to communicate directly or indirectly with the other devices, such as an external user interface or other computing devices. Additionally, computing device can include hardware entities 3314, such as microprocessors, application specific integrated circuits (ASICs), and other hardware. As shown in FIG. 33, the hardware entities 3314 can also include a removable memory unit 3316 comprising a computer-readable storage medium 3318 on which is stored one or more sets of instructions 3320 (e.g., software code) configured to implement one or more of the methodologies, procedures, or functions described herein. The instructions 3320 can also reside, completely or at least partially, within the memory 3316 and/or within the CPU 3306 during execution thereof by the computing device 3300. The memory 3316 and the CPU 3306 also can constitute machine-readable media.

While the computer-readable storage medium 3318 is shown in an exemplary embodiment to be a single storage medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure.

The term “computer-readable storage medium” shall accordingly be taken to include, but not be limited to solid-state memories (such as a memory card or other package that houses one or more read-only (non-volatile) memories, random access memories, or other re-writable (volatile) memories), magneto-optical or optical medium (such as a disk or tape). Accordingly, the disclosure is considered to include any one or more of a computer-readable storage medium or a distribution medium, as listed herein and to include recognized equivalents and successor media, in which the software implementations herein are stored.

System interface 3322 can include a network interface unit configured to facilitate communications over a communications network with one or more external devices. Accordingly, a network interface unit can be provided for use with various communication protocols including the IP protocol. Network interface unit can include, but is not limited to, a transceiver, a transceiving device, and a network interface card (NIC).

As noted above, those skilled in the art will recognize that such a plasma reactor may not only be used with conventional gas treatment, but also for decontamination, odor control, etc. While the description above refers to particular embodiments of the invention, it will be understood that many modifications may be made without departing from the spirit thereof. The accompanying claims are intended to cover such modifications as would fall within the true scope and spirit of the invention.

Applicants present certain theoretical aspects above that are believed to be accurate that appear to explain observations made regarding embodiments of the invention. However, embodiments of the invention may be practiced without the theoretical aspects presented. Moreover, the theoretical aspects are presented with the understanding that Applicants do not seek to be bound by the theory presented.

While various embodiments of the invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the invention should not be limited by any of the above described embodiments. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

1. A method for the treatment of a gas, comprising:
providing the at least one device comprising first and second dielectric plates facing each other and defining a discharge region, at least one first electrode, at least one second electrode, and a conductive layer, the at least one first electrode and the at least one second electrode each comprising elongate electrodes disposed on an inner surface of the first dielectric plate and arranged substantially in parallel, the conductive layer disposed beneath the inner surface and extending over at least the portion of the first dielectric plate between the at least one first electrode and the at least one second electrode, and the conductive layer electrically coupled to the at least one second electrode and electrically isolated from the at least one first electrode; and
directing a first gas into at least one device;
generating a plurality of voltage pulses between the at least one first electrode and the at least one second electrode to generate a substantially non-thermal plasma for the first gas in the discharge region to yield a second gas; and
directing the second gas from the at least one device,
wherein the generating comprises selecting a voltage, a repetition rate, and a pulse width for the plurality of voltage pulses based on the type of gas, a thickness and a permittivity of the first dielectric plate and a gap between the at least one first electrode and the at least one second electrode, and wherein a pressure in the discharge chamber is substantially atmospheric pressure.

2. The method of claim 1, wherein the first gas comprises steam.

3. (canceled)

4. The method of claim 1, wherein the generating comprises selecting the voltage for the plurality of voltage pulses to be between about 100V and about 300 kV.

5-6. (canceled)

7. The method of claim 1, wherein the generating comprises selecting the pulse repetition rate to be between about 1 Hz and about 10000 Hz.

8-9. (canceled)

10. The method of claim 1, wherein the generating comprises selecting the pulse width to be between about 1 ns and about 1000 ns.

11-13. (canceled)

14. The method of claim 1, further comprising:
selecting the first gas to be a mixture of steam and benzene;
selecting the plurality of voltage pulses to cause the non-thermal plasma to generate radicals from the steam that react with at least a portion of the benzene to produce phenol in the second gas;
condensing the second gas to generate a liquid;
distilling the liquid to generate liquid phenol and a third gas comprising steam and benzene.

15. (canceled)

16. The method of claim 1, further comprising:
selecting the first gas to be a mixture of tritium-contaminated heavy water molecules and hydrogen containing deuterium; and
selecting the plurality of voltage pulses to cause the non-thermal plasma to result in a hydrogen isotopic exchange between the tritium-contaminated heavy water molecules and the hydrogen containing deuterium.

17. A system, comprising:
at least one device comprising:
one or more dielectric portions defining at least one elongate and substantially continuous inner surface with an inlet and an outlet;
at least one first electrode, at least one second electrode, and at least one a conductive layer, the at least one first electrode and the at least one second electrode disposed on the inner surface and the at least one conductive layer beneath the inner surface and substantially surrounding a discharge region defined by the inner surface, the at least one conductive layer electrically coupled to the at least one second electrode and electrically isolated from the at least one first electrode; and
a circuit in communication with the at least one device and configured for generating a plurality voltage pulses between the at least one first electrode and the at least one second electrode.

18. The system of claim 17, wherein each of the at least one first electrode and the at least one second electrode comprise elongate electrodes disposed on the inner surface parallel to each other.

19-20. (canceled)

21. The system of claim 17, wherein the inner surface comprises a substantially cylindrical surface.

22. The system of claim 17, wherein the at least one device comprises a plurality of devices, wherein a first of the plurality of devices is disposed with the discharge region of a second of the plurality of devices, and wherein the at least one conductive layer of the second of the plurality of devices is not exposed to the discharge region of the first of the plurality of devices.

23. The system of claim 17, wherein the at least one device comprises a plurality of devices, and wherein the discharge region of first of the plurality of devices is connected in parallel with the discharge region of a second of the plurality of devices.

24. The system of claim 17, wherein the at least one device comprises a plurality of devices, and wherein the discharge region of first of the plurality of devices is connected in series with the discharge region of a second of the plurality of devices.

25. A system for the treatment of a surface, comprising:
a first dielectric portion with a first inner surface and a first outer surface;
a second dielectric portion with a second inner surface and a second outer surface, the second dielectric portion disposed adjacent to the first dielectric portion such that the first inner surface faces the second inner surface and defines a discharge region;
a first electrode disposed at an inlet end of the discharge region on the first inner surface;
at least one second electrode disposed at an outlet end of the discharge region, the at least one second electrode disposed on at least one of the first inner surface and the second inner surface;
at least one conductive layer extending over the first outer surface and the second outer surface, the at least one conductive layer electrically coupled to the at least one second electrode and electrically isolated from the at least one first electrode;
a circuit in communication with the at least one device and configured for applying a series of voltage pulses between the at least one first electrode and the at least one second electrode.

26. The system of claim 25, further comprising a slit cover coupled to the outlet end having at least one slit.

27-28. (canceled)

29. The system of claim 25, wherein the first dielectric portion and the second dielectric portion are substantially rectangular dielectric plates, the dielectric plates arranged substantially in parallel and substantially overlapping each other.

30. (canceled)

31. The system of claim 25, further comprising a source of air or air mixed with other gases coupled to the inlet end.

32. The system of claim 25, further comprising a source of steam or steam mixed with other gases coupled to the inlet end.

33. The system of claim 25, wherein the voltage pulses delivered by the circuit are between 100V and 500 kV.

34. The system of claim 25, wherein the pulse width is between 1 ns and 1000 ns

35. The system of claim 25, wherein the voltage pulses delivered by the circuit are delivered with a repetition rate between 1 Hz and 1000 Hz.

36-41. (canceled)
